(19) United States
(12) Patent Application Publication
(10) Pub. No.: US 2023/0233500 A1
(43) Pub. Date: Jul. 27, 2023

(54) Gasdermin D (GSDMD) Succination for the Treatment of Inflammatory Disease

(71) Applicant: University of Massachusetts, Boston, MA (US)

(72) Inventors: Katherine A. Fitzgerald, Wellesley, MA (US); Fiachra Humphries, Shrewsbury, MA (US); Paul Thompson, Wellesley, MA (US)

(21) Appl. No.: 17/927,642

(22) PCT Filed: Jun. 11, 2021
(86) PCT No.: PCT/US2021/037037

§ 371 Date: Nov. 23, 2022

Related U.S. Application Data
(60) Provisional application No. 63/088,854, filed on Oct. 7, 2020.
(60) Provisional application No. 63/038,000, filed on Jun. 11, 2020.

(51) Int. Cl. A61K 31/225; A61K 31/7105; A61K 31/4015; A61P 29/00

(52) U.S. Cl. CPC A61K 31/225; A61K 31/7105; A61K 31/4015; A61P 29/00

(57) ABSTRACT

Described herein are compositions and methods for treating inflammation, and inflammation-related conditions, using fumarate or fumarate analogs or inhibitors of fumarate hydratase (FH).

### CLAIM OF PRIORITY

This application claims the benefit of U.S. Provisional Patent Application Serial Nos. 63/038,000, filed on Jun. 11, 2020, and 63/088,854, filed on Oct. 7, 2020. The entire contents of the foregoing are hereby incorporated by reference.

### TECHNICAL FIELD

Described herein are compositions and methods for treating inflammation, and inflammation-related conditions, using fumarate or fumarate analogs or inhibitors of fumarate hydratase (FH).

### BACKGROUND

Dynamic transcriptional responses as well as cell death pathways including apoptosis, necroptosis and pyroptosis contribute to the host response to infection. Pyroptosis is a noxious form of cell death with danger associated molecular patterns (DAMPs) released from dying cells that propagate inflammation to curb pathogen spread (Broderick et al., Annual Review of Pathology: Mechanisms of Disease 10, 395-424 (2015)). In the case of excessive cell death, these DAMPs drive inflammatory diseases (Broderick et al., Annual Review of Pathology: Mechanisms of Disease 10, 395-424 (2015); Swanson et al., Nat Rev Immunol. 2019 August; 19(8):477-489). Pyroptosis is dependent on caspase-mediated cleavage of gasdermin D (GSDMD). Caspase cleavage of GSDMD liberates an N-terminal pore forming fragment (GSDMD-N, p30), which oligomerizes and inserts into the plasma membrane forming pores that serve as a conduit for the release of IL-18, IL-18 and ultimately the demise of the cell (Kayagaki et al., Nature 526, 666 (2015); Shi et al., Nature 526, 660-665 (2015)).

### SUMMARY

Provided herein are methods for treating a subject who has an inflammatory condition, the method comprising administering to the subject an effective amount of (i) fumarate or a fumarate analog, or (ii) an inhibitor of fumarate hydratase (FH). Also provided are compositions comprising or consisting of (i) fumarate or a fumarate analog, or (ii) an inhibitor of fumarate hydratase (FH) as an active agent, for treating a subject who has an inflammatory condition.

In some embodiments, the inflammatory condition is associated with or a result of a viral infection, e.g., infection with SARS-CoV-2.

In some embodiments, the inflammatory condition is septic shock, allergy, asthma, autoimmune diseases, cancer, celiac disease, glomerulonephritis, hepatitis, inflammatory bowel disease, preperfusion injury, transplant rejection, myalgic encephalomyelitis/chronic fatigue syndrome, neonatal-onset multisystem inflammatory disease (NOMID), or Familial Mediterranean Fever (FMF).

In some embodiments, the fumarate or fumarate analog is Dimethyl Fumarate, Diroximel Fumarate, or Tepilamide Fumarate.

In some embodiments, the inhibitor of FH is a small molecule inhibitor or an inhibitory nucleic acid targeting a nucleic acid encoding FH. In some embodiments, the inhibitory nucleic acid is an antisense RNA, antisense DNA, chimeric antisense oligonucleotides, antisense oligonucleotides comprising modified linkages, interference RNA (RNAi), short interfering RNA (siRNA), or a short hairpin RNA (shRNA).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-L. Fumarate inhibits pyroptosis. Bone marrow-derived macrophages (BMDMs) were treated as indicated and LDH, IL-1β or TNF-α measured by ELISA (A-C), GSDMD/β-actin by immunoblotting (D) or permeabilization of cells to SYTOX orange (E-F). GSDMD oligomerization in native and reduced cell lysates treated as above (G). Timecourse of permeabilization to SYTOX orange or GSDMD-N formation in cells treated as indicated (H-I). Survival rates of WT mice after 50 mg/kg of LPS (n−10) or LPS+fumarate (n=9) (J). Levels of IL-1β or TNF-α from serum of WT mice at 5 hours post-intraperitoneal injection with 5 mg/kg LPS (n=6) or control PBS (n=3) (K, L). A, B, C, I and are pooled from three independent experiments. D, E, F, G, H and I are representative from three independent experiments. J, K, L data points indicate individual mice. *, P<0.05, **, P<0.01; ***, P<0.001; ****, P<0.0001) (J, Mantel Cox survival analysis; A-C, K Two-way ANOVA). Error bars show means±SEM.

FIGS. 2A-J. Succination of GSDMD inhibits pyroptosis. Immunoblot of GSDMD in streptavidin pulldown from clicked lysates treated as indicated (A-B). Representative MS spectra of 2-monomethyl and 2-dimethylsuccination of GSDMD immunoprecipitated from fumarate treated BMDMs (C-D). Immunoblot of GSDMD in clicked lysates from transfected HEK293T. Cell death of HEK293T transfected as indicated (F-H). Immunoblots of casplp20 and GSDMD in BMDMs (I). Immunoblots of an in vitro binding assay of succinated and non-succinated GSDMD incubated with caspase 1 beads (J). A, B, F, J, K representative images from three independent experiments. C, D, E, representative mass spec spectra from two independent experiments. H, I pooled data from three independent experiments. ***, P<0.001 (One-way anova). Error bars show means±SEM.

FIGS. 3A-J. Fumarate targets GSDMD and Gasdermin E (GSDME). Kinetic cell death of WT and Gsdmd-BMDMs (A) or WT/GSDMD^{-/-} THP1 cells (B) treated as indicated. LDH and IL-1β release from WT and Gsdmd^{-/-} BMDMs (C-D). Immunoblot of GSDMD and GSDME in native and reduced cell lysates from BMDMs (E). Immunoblot of GSDME in streptavidin pulldown from clicked lysates (F). Representative Mass spectra of succinated GSDME (G-H). Immunoblot analysis of GSDMD and GSDME from WT and Gsdmcd^{-/-} BMDMs (I). A, B representative of three independent experiments. C, D, I pooled data from three independent experiments. E, F, J representative images from 3 independent experiments. ***, P<0.0001, * P<0.00001 (Two-way ANOVA). Error bars show means±SEM.

FIGS. 4A-L. Succination of GSDMD alleviates experimental autoimmuine encephalopathy (EAE) and multiple sclerosis (MS). Clinical scores of WT mice administered vehicle or fumarate daily after EAE induction, respectively (n=10). Representative H&E, LFB and GSDMD staining (B) and pathology evaluation (C) of spinal cord sections from mice showing inflammatory cell infiltration and demyelination, respectively. Scale bar: 200 m. Immunoblot of GSDMD in spinal cord tissue (D). Flow-cytometric analysis of CD45⁺, CD45⁺CD4⁺ T cells, CD45⁺CD8⁺ T cells, and CD45⁺CD11b⁺ monocytes infiltrated to the spinal cord and brain of the mice in (A) (n=5) (E-F). Th1 (IFN-γ⁺) and Th17 (IL-17A⁺) cells from CD4⁺ T cells (n=5) (G-H). IHC staining of GSDMD-N in post-mortem lesions from MS patients (J). IL-1β levels in serum from healthy controls (n=6), MS patients (MS, n=9) and MS patients receiving Tecfidera delayed release capsules (n=9) (K). Immunoblot of GSDMD-N(L) and densitometry analysis of GSDMD-N in PBMCs from MS (n=8) or PMS Tecfidera (n=3) (J-K). *, P<0.05; **, P<0.01; ***, P<0.001 (A, Mann-Whitney U test; C, students t-test; E-H, multiple t-test) Error bars show means±SEM.

FIGS. 5A-G. Fumarate inhibits pyroptosis. (A) SYTOX Orange and Hoechst stained WT BMDMs treated with LPS for 2 hours, DMSO (vehicle) or fumarate (25 μM) for 1 hour and nigericin for 6 hours. (B) LDH assay conditioned medium from WT BMDMs treated with LPS for 3 hours, nigericin for the indicated times and DMSO (vehicle) or fumarate (25 μM) 15 minutes after each nigericin (10 μM) timepoint. (C) Immunoblot analysis of GSDMD in BMDMs treated as in (C). (D-E) LDH (D) and immunoblot analysis of GSDMD and β-actin in lysates (E) from WT BMDMs treated with DMSO (vehicle) or fumarate (25 M) for 1 hour followed by salmonella infection (MOI 10) for 6 hours. (F-G) LDH (F) and immunoblot analysis of GSDMD and β-actin in lysates (G) from WT BMDMs primed with LPS for 2 hours, followed by treatment with fumarate (25 μM) transfection with Poly (dA:dT) (1 μg). A, C, E, G are representative of 3 independent experiments. B, D, F are pooled data from 3 independent experiments. ***, P<0.001, ****, P<0.00001 (Two-way ANOVA). Error bars show means±SEM.

FIGS. 6A-G. Fumarate inhibits pyroptosis in Human cells. (A-B) Representative images (A) and kinetic data (B) of SYTOX Orange and Hoechst-stained PMA-differentiated THP1 cells treated with LPS for 2 hours, DMSO (vehicle) or fumarate (25 μM) for 1 hour and nigericin (10 μM) for 6 hours. (C) LDH release from (A). (D-E) Kinetic cell death data of SYTOX Orange and Hoechst stained (D) or LDH assay of conditioned medium from primary CD14+ positive monocytes treated with LPS for 2 hours DMSO (vehicle) or fumarate (25 μM) for 1 hour and nigericin (10 μM) for 6 hours. (F) LC/MS quantification of fumarate in WT BMDMs treated with LPS for 2 hours, FHIN1 for 1 hour and nigericin for 1 hour. (G) Representative images of WT primary BMDMs stained with SYTOX Orange and Hoechst following treatment with LPS for 2 hours, DMSO (vehicle) or fumarate (25 μM) or FHIN1 (25 μM) for 1 hour and nigericin for a timecourse of 6 hours. A, B, D, G are representative of 3 independent experiments. C, E, F are pooled data from 3 independent experiments. ***, P<0.001, ****, P<0.00001 (Students t-test). Error bars show mean±SD (B, D) or SEM (C, E).

FIGS. 7A-K. Fumarate regulates pyroptosis independently of NRF2 and GAPDH. (A-B) Kinetic cell death data of WT primary BMDMs treated with LPS for 2 hours, DMSO (vehicle) or fumarate (25 μM) or heptilidic acid (HA) (0.5 μM or 1 μM) (A) or ML385 (1 μM) (B) followed by nigericin (10 μM) for 6 hours. (C-D) LDH (C) and IL-1B (D) release from WT primary BMDMs treated with LPS for 2 hours, DMSO (vehicle) or fumarate (25 μM) for 1 hour, HA or ML385 (1 μM) for 1 hour followed by nigericin (10 μM) or 1 hour. (E-F) Immunoblot analysis of GSDMD in cell lysates from WT BMDMs treated as in (B) with the indicated inhibitors. (G-H) QPCR (G) and immunoblot (H) analysis of iNos in WT BMDMs treated with DMSO (vehicle) or HA (1 μM) for 1 hour and LPS for 3 (G) or 6 (H) hours. (I) QPCR analysis of Hmox1 mRNA in WT BMDMs treated with DMSO (vehicle) or ML385 (1 μM) for 3 hours. (J-K) LDH assay (J) and immunoblot analysis of GSDMD (K) in WT BMDMs transfected with control non-targeting siRNA or NRF2-specific siRNAs (50 nM) for 48 hours followed by treatment with LPS for 2 hours, DMSO (vehicle) or fumarate (25 μM) and nigericin (10 μM) or 1 hour. A, B, E, F, H, K representative images from 3 independent experiments. C, D, G, I, K pooled data from 3 independent experiments. *, P<0.01, ****, P<0.00001 (Two-way ANOVA). Error bars show means±SD (A, B) or SEM (C, D, G, I, J).

FIGS. 8A-B. Fumarate inhibits pyroptosis in vivo. (A-B) LC/MS Fumarate quantification in splenocytes (A) and IL-1 ELISA analysis (B) in serum from WT C57/BL6 mice administered FHIN2 (50 mg/kg) by IP injection followed by LPS (10 mg/kg) by IP injection for 5 hours. Data points indicative of individual mice. **, P<0.01 (Two-way ANOVA). Error bars show means±SEM.

FIGS. 9A-D. Synthesis of MMF-Yne probe. (A) Structures of MMF-Alkyne, fumarate and MMF. (B) ESI-MS spectrum of MMF-Yne. (C-D) LDH (C) and IL-1B (D) release from WT BMDMs treated with LPS for 2 hours with DMSO (vehicle) or fumarate (25 μM) for 1 hour, MMF (25 μM) or the MMF-Alkyne (25 μM) probe followed by treatment with nigericin (10 μM) for 1 hour. C, D pooled data from 2 independent experiments. Error bars show means±SEM.

FIG. 10. Illustrative schematic of click-chemistry based proteomics. Schematic representation of MMF-Yne proteomics. Primary WT BMDMs were treated with LPS for 2 hrs, MMF-Yne for 1 hour and nigericin (10 μM) for 1 hour. Lysates were subsequently Cu2+ clicked using a biotin-azide and streptavidin beads for pulldown of MMF-Yne interacting proteins. Streptavidin beads were digested using trypsin, reduced and alkylated and subjected to mass spec analysis.

FIGS. 11A-E. Fumarate binds to GSDMD. (A) Immunoblot analysis of MMF-Yne bound proteins using a streptavidin-IR dye. (B) Representative MS spectra of GSDMD identified in streptavidin pulldowns subjected to mass spec. (C-D) Immunoblot analysis (C) and densitometry analysis (D) of streptavidin-IR dye detecting MMF-Yne labelled recombinant GSDMD incubated with the indicated concentrations of fumarate and copper clicked using a biotin-azide. (E) Intact MS analysis of recombinant GSDMD (1 μM) incubated in a succination reaction with DMSO (vehicle) or fumarate (25 μM) for 2 hours. A-E Representative images from 2 independent experiments..

FIGS. 12A-B. Succination of GSDMD by endogenous fumarate (A) Representative MS spectra of 2-succinyl succination on Cys434 of GSDMD immunoprecipitated from WT BMDMs treated with FHIN1 (50 μM) for 1 hour, LPS for 2 hours and nigericin (10 μM) for the indicated times. (B) Immunoblot of 2-succinyl-cysteine and GSDMD in immunoprecipitates (IP) and lysates (input) from WT and GSDMD −/− BMDMs. Representative experiments.

FIGS. 13A-B. Fumarate inhibits GSDMD and GSDME comparably. (A) Kinetic cell death data of WT (left-panel) and GSDMD−/− (right-panel) primary BMDMs with LPS for 2 hours, DMSO (vehicle) or fumarate (0-50 μM) followed by nigericin (10 μM) for 6 hours. (B) Immunoblot analysis of GSDMD, GSDME and -actin in cell lysates from WT and GSDMD−/− BMDMs treated as in (A).

FIGS. 14A-C. Fumarate analogues inhibit pyroptosis (A) Kinetic cell death data of WT primary BMDMs (A) and THP1 cells (B) stained with SytoxOge and Hoechst following treatment with LPS for 2 hr, DMSO (Vehicle), Fumarate (Fum), Monomethyl Fumarate (MMF), Diroximel Fumarate (Dirox) or Tepilamide (Tepi), for 1 hour and nigericin for the indicated time course. (C) Immunoblot analysis of GSDMD cell lysates from WT BMDMs treated as in (A) with the indicated fumarate analogues (25 μM). A-C are representative of 3 independent experiments.

FIGS. 15A-E. Fumarate alleviates FMF. (A-D) Weight gain (A), spleen weight (B), ELISA of serum IL-1B and immunoblot of spleen GSDMD (D) liver H&E (E) from Mefv726/726 receiving vehicle diet or a diet containing dimethyl fumarate (100 mg/kg/day) for 6 weeks. Data points indicative of individual mice. *, P<0.05 ***, P<0.001 (t-test or Two-way ANOVA). Error bars show means±SEM.

FIG. 16. Proposed mechanism of fumarate mediated regulation of Pyroptosis. Mitochondrial membrane rupture during pyroptosis results in accumulation of fumarate in the cytosol. Fumarate released during inflammasome activation results in the succination of Cys192 in gasdermin-d. Succination of GSDMD results in impaired GSDMD oligomerization and pyroptosis.

### DETAILED DESCRIPTION

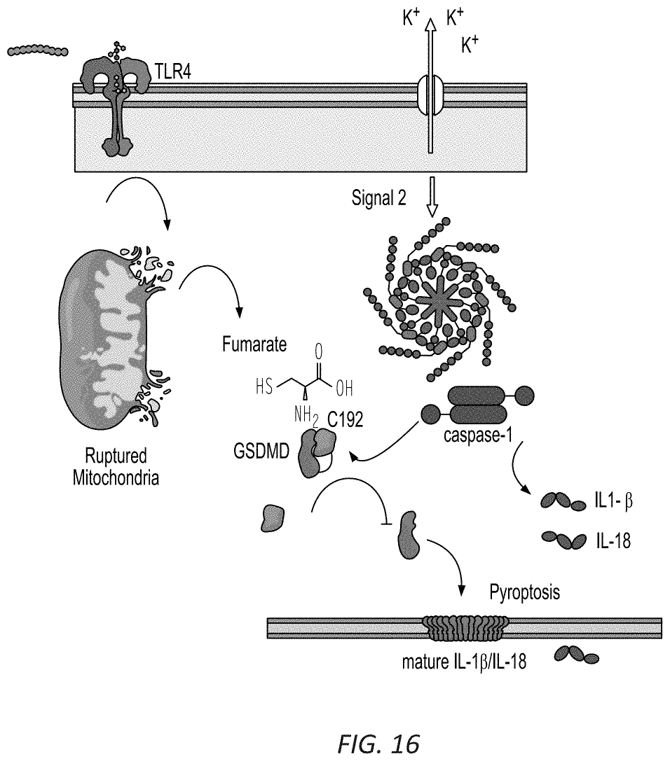

As demonstrated herein, fumarate is a key regulator of pyroptosis. Cys¹⁹² in the gasdermin-d N-terminal pore forming fragment is the predominant target site. Succination of GSDMD on Cys192 prevents its processing and oligomerization which limits pore formation, cytokine release and cell death (FIG. 16). Several studies have identified small-molecule inhibitors that target Cys¹⁹¹/Cys¹⁹² in gasdermin-d, highlighting this functional cysteine as a hub for targeted inhibition (15, 20, 21). Indeed, Succination of gasdermin-d at Cys¹⁹² inhibits oligomerization however other succination sites in gasdermin-d, Cys⁵⁷/Cys⁷⁷ may also play a role in affecting protein function through protein-protein interactions such as with Caspase1. Our studies also provide new mechanistic insight into the immunomodulatory activity of Tecfidera (fumarate), the frontline treatment for MS. Not only do these studies shed new light on the mechanism of action of this drug but also underscore the importance of gasdermin-d as a driver of neuroinflammation in this disease.

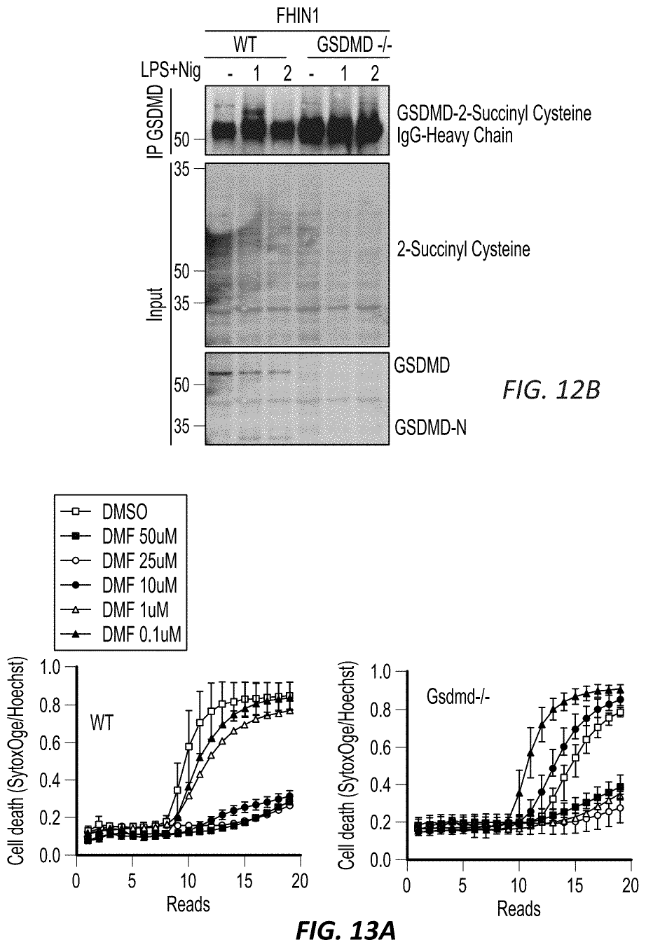

Further, our data also support a model where endogenous fumarate serves to limit gasdermin-d-dependent cell death. The targeting of Cys¹⁹² by a cell intrinsic succination mechanism has not been shown previously We propose a model where the accumulation of fumarate in activated cells serves as a negative feedback loop to terminate cell death by inducing the succination and inactivation of gasdermin-d, thus limiting the release of cytokines and DAMPs from dying cells (FIG. 12A-B). The ESCRT machinery has been described as a key regulator of gasdermin-d pore formation by direct repair of membrane pores (25). Little is known however about mechanisms that regulate and limit gasdermin-d directly. Endogenous fumarate released from mitochondria during initiation of pyroptosis could modify gasdermin proteins and limit cell death. Our identification of fumarate as a suppressor of pyroptosis via the post-translational modification succination, provides new insight into the use of fumarate-based therapeutics for the treatment of neuroinflammation and other chronic inflammatory diseases.

Collectively, the data presented herein indicate that fumarate mitigates pyroptosis.

Methods of Treatment

The methods described herein include methods for the treatment of inflammatory diseases (also referred to herein as disorders) driven by pyroptosis and Gasdermin D. Gasdermin-induced pyroptosis plays a role in a number of hereditary diseases and inflammatory/autoinflammatory disorders as well as in cancer (Broz et al., Nature Reviews Immunology 20:-157(2020)). In some embodiments, the disorder is septic shock, allergy, asthma, autoimmune diseases, cancer, celiac disease, glomerulonephritis, hepatitis, inflammatory bowel disease, preperfusion injury, transplant rejection, myalgic encephalomyelitis/chronic fatigue syndrome, neonatal-onset multisystem inflammatory disease (NOMID) (Xiao et al., PLoS Biol. 2018 Nov. 2; 16(11):e3000047), or Familial Mediterranean Fever (FMF) (Kanneganti et al., J Exp Med. 2018 Jun. 4; 215(6): 1519-1529); see also Orning et al., J. Exp. Med. 2019 Vol. 216 No. 11 2453-2465. In some embodiments, the disorder is inflammation secondary to an infection, e.g., post-viral inflammation syndrome (e.g., post-viral fatigue syndrome) or post-viral multisystem inflammatory syndrome. In some embodiments, the disorder is inflammation secondary to a coronavirus infection, e.g., SARS-CoV-2 infection, e.g., a multisystem inflammatory syndrome in children or adults. See, e.g., Bannister, Postgraduate Medical Journal (1988) 64, 559-567; Cheung et al., JAMA. 2020; 324(3):294-296. Published online Jun. 8, 2020.

Generally, the methods include administering a therapeutically effective amount of fumarate or fumarate analogues as described herein, to a subject who is in need of, or who has been determined to be in need of, such treatment.

As used in this context, to “treat” means to ameliorate at least one symptom of the inflammatory diseases driven by pyroptosis and Gasdermin D. Administration of a therapeutically effective amount of a compound described herein for the treatment of an inflammatory diseases driven by pyroptosis and Gasdermin D will result in decreased inflammation and inflammation-related symptoms or phenotypes.

In some embodiments, the disorder is not multiple sclerosis (MS),

Fumarate and Fumarate Analogues

Compounds useful in the methods described herein include fumarates and fumarate analogues. Examples include Dimethyl Fumarate (e.g., TECFIDERA), Diroximel Fumarate and Tepilamide Fumarate. See also Hsieh et al., PLoS One. 2014; 9(6): e98385. The analogs must also succinate gasdermin-d.

Inhibitors of Fumarate Hydratase

The present methods can also or alternatively include administration of an inhibitor of fumarate hydratase. Small molecule inhibitors are known in the art, see, e.g., Takeuchi et al., J Am Chem Soc. 2015 Jan. 21; 137(2):564-7; Kasbekar et al., PNAS Jul. 5, 2016 113 (27) 7503-7508; Whitehouse et al., J Med Chem. 2019 Dec. 12; 62(23):10586-10604.

Alternatively, an inhibitory nucleic acid targeting Fumarate Hydratase (FH) can be used. Exemplary target sequences for human FH are provided in GenBank at Acc. No. NM_000143.4 (nucleic acid) and NP_000134.2 (protein). Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); or a short, hairpin RNA (shRNA); or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Orom et al., Gene. 2006 May 10; 372( ):137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH, ˜N(CH3)˜O˜CH2 (known as a methylene(methylimino) or MMI backbone], CH2-O—N(CH3)-CH2, CH2-N(CH3)-N (CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Pharmaceutical Compositions and Methods of Administration

The methods described herein include the use of pharmaceutical compositions comprising or consisting of fumarate or fumarate analogues as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., other anti-inflammatory agents, e.g., a monoclonal antibody targeting an inflammatory cytokine, e.g., anti-Interleukin 1 (IL-1), e.g., Canakinumab, or colchicine (e.g., for FMF). Alternatively, no other active agents may be present or used.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### EXAMPLES

The invention is further described in the following examples, which do not limit the scope of the invention described in the claims.

### Materials and Methods

The following materials and methods were used in Example 1 below.

Mice

All animal experiments were approved by the Institutional Animal Care Use Committees at the University of Massachusetts Medical School. Animal were kept in specific pathogen free (SPF) environment. Male and female C57/BL6 and Gsdmd−/− mice were used. Gsdmd−/− mice were generated as previously described (22). MefvV726/V726 mice were a gift from Prof. Daniel Kastner (23). Sample sizes used are in line with other similar published studies. All animals were used at age 8-12 weeks.

Reagents All chemicals and metabolites were purchased from sigma. Metabolites were dissolved in DMSO and used at the follow concentrations based on previous published studies (7, 8,9): alpha-ketoglutarate (1 mM), succinate (5 mM), methyl/ethyl pyruvate (5 mM), diethyl butyl malonate (1 mM), dimethyl fumarate (25 μM), monomethyl fumarate (25 μM), 4-octyl itaconate (50 μM) triethyl citrate (10 mM). Nigericin (N7143) was purchased from sigma. LPS (ALX-581-010-L002) was from enzo. Cells were transfected using genejuice according to the manufacturer's instructions.

Induction and Assessment of Experimental Autoimmune Encephalomyelitis (EAE)

EAE was induced using MOG₃₅₋₅₅ peptide (200 μg per mouse) emulsified with CFA (50 μl per mouse, including 4 mg/ml M. tuberculosis H37Ra) and 50 μl of incomplete Freund's adjuvant. 250 ng of Pertussis toxin was administered IV on day 0 and 2 post immunization. Mice were scored daily for clinical signs of EAE in a blinded fashion. EAE score was calculated as follows: 0.5, partial tail paralysis; 1, tail paralysis; 1.5, reversible corrective reflex impairment; 2, corrective reflex impairment; 2.5, one hindlimb paralysis; 3, both hindlimbs paralysis; 3.5, both hindlimbs paralysis and one forelimb paralysis; 4, hindlimb and forelimb paralysis; and 5, death.

Histological and Immunohistochemistry

Tissue blocks were section at 5 μm thick. For paraffin-embedded tissue, CNS tissue was rapidly dissected from mice perfused with PBS. Tissues were fixed in 4% paraformaldehyde overnight. Tissue sections were stained with H&E for evaluation of inflammation. IHC staining of gasdermin-d was performed was detected using horseradish peroxidase-conjugated secondary antibodies after heat-induced antigen retrieval. Diaminobenzidine was used for detection. Images were captured with a Nikon 50i microscope.

Flow Cytometry

For analysis of infiltrating immune cells CNS, brains, and spinal cords from MOG₃₅₋₅₅-immunized mice were excised and digested at 37° C. with collagenase type IV (0.5 mg/ml; Sigma-Aldrich) and DNase I (10 U/ml; Roche) in RPMI 1640 under agitation (200 rpm) conditions for 1 hour. Following digestion tissues were filtered through a 100-μm filter. Cell suspensions separated using a Percoll density gradient (GE Healthcare) and separated by collecting the interface fractions between 37% and 70% Percoll. Mononuclear cells were isolated from the interface. The cells were suspended in PBS containing 2% (wt/vol) FBS. Cells were washed 3 times and stained with cell surface marker antibodies for FACS analysis. The following antibodies were used: Anti-CD45-FITC (30-F11,11-0451-82), anti-CD4-FITC (GK1.5, 11-0041-82), anti-CD8a-PE (53-6.7,12-0081-83), anti-CD11b-APC (M1/70,17-0112-82), anti-IL17A-PE (eBio17B7,12-7177-81), anti-IFN-γ-APC (XMG1.2,17-7311-81), Fixable Viability Dye eFluor™ 506 (65-0866-14), and Intracellular Fixation & Permeabilization Buffer Set Kit (85-88-8824-00) were from eBioscience. Anti-CD4-APC-Cy7 (GK1.5,100414) was from Biolegend. All flow cytometry was performed on an Attune NxT flow cytometer (Thermo Fisher Scientific), and data were analyzed by FlowJo 7.6.1 software.

Plasmids

All GSDMD constructs were purchased from addgene. Full length FLAG-GSDMD (#80950), FLAG-GSDMD-NT (#80951), FLAG-GSDMD-NT-C192A (#133891).

Cell Culture

Human kidney cell line HEK293T was cultured in Dulbecco's modified Eagle's medium supplemented with 10% (v/v) fetal bovine serum, 100 U/ml penicillin and 100 g/ml streptomycin. Human peripheral blood monocyte cell line, THP1 cells were cultured in RPMI-1640 medium supplemented with 10% (v/v) fetal bovine serum, 100 U/ml penicillin and 100 g/ml streptomycin. For isolation of BMDMs, tibias and femurs were removed from wild type and GsdmD^{-/-} mice and bone marrow was flushed with complete DMEM-medium. Cells were plated in medium containing 20% (v/v) conditioned medium of L929 mouse fibroblasts cultured for 7 days at 37° C. in a humidified atmosphere of 5% C02. Medium was replaced every 3 days.

ELISA

Conditioned media or serum was collected as indicated and IL-1β or TNF-α, were quantified by sandwich ELISA (R&D Systems).

Cell Death Assays

Cells were plated, treated and incubated in phenol red free medium. Following treatment medium was collected and assessed for LDH release using the CytoTox96 non-radioactive cytotoxicity assay (Promega) as per manufacturer's instructions. Kinetic cell death assays were performed using SytoxOrange and the Biotek Cytation5.

Inflammasome Activation Assays

Cells were primed with LPS (100 ng/ml) from Escherichia coli serotype EH100 (ra) TLRgrade for 3 h followed by stimulation with the inflammasome activator Nigericin (20 M) for 1 h. For noncanonical inflammasome activation, cells were primed with 1 μl g/ml Pam3CSK4 (Invivogen) for 2 h, followed by transfection of LPS (2 μg/ml) using Lipofectamine 2000 for 16 hours.

Immunoblotting and Immunoprecipitation

Primary BMDMs from WT or GsdmD^{-/-} mice were cultured in 12-well plates (1×10⁶ cells per ml; 1 ml) or 10 cm dishes (2×10⁶ cells per ml; 10 ml). HEK293T cells (2.5×10⁵ cells per ml; 3 ml) were cultured in 6-well plates and transfected with FLAG-tagged GSDMD constructs where indicated. For cell lysate analysis cells were lysed directly in 1× Lamelli sample buffer. For native gel analysis cells were lysed in in NP-40 lysis buffer (50 mM Tris-HCl, pH 7.4, containing 150 mM NaCl, 0.5% (w/v) IgePal, 50 mM NaF, 1 mM Na3VO4, 1 mM dithiothreitol, 1 mM phenylmethylsulfonyl fluoride and protease inhibitor cocktail. For immunoprecipitation of GSDMD, cells were treated as indicated and then collected in 500 μl RIPA buffer, followed by incubation for 15 min on ice. Lysates were incubated with GSDMD antibody and protein A-protein G-agarose was added to each sample, followed by incubation overnight at 4° C. Immunoprecipitates were collected by centrifugation and washed four times with 1 ml of RIPA buffer. Immunprecipates were eluted from beads using 1× sample buffer. Samples were resolved by SDS-PAGE and stained using simply blue safe stain or transferred to nitrocellulose membranes and analyzed by immunoblot. Immunoreactivity was visualized by the Odyssey Imaging System (LICOR Biosciences). For immunoblotting of IL-1β in cell supernatants, conditioned medium was collected and filtered using filter spin columns to reduce salt and remove abundant serum proteins. Filtrates were added to 4×SDS-PAGE sample buffer and resolved by SDS-PAGE for immunoblot analysis. GSDMD (ab209845) and GSDME antibodies were from abcam (ab215191). Caspase 1 antibody was from adipogen. Fumarase antibody was from cell signalling and anti-β-actin (AC-15; A1978) were from Sigma; anti-mouse IRDye™ 680 (926-68070) and anti-rabbit IRDye™ 800 (926-32211) were from LI-COR Biosciences.

Intact Protein Analysis

Intact mass analysis of GSDMD protein was carried out using an Acquity UPLC (Waters Corporation, Milford, Mass.) coupled to a Synapt G2-Si (Waters) quadrupole time-of-flight mass spectrometer fitted with an electrospray ionization source. Liquid chromatography was carried out with a 3.0×50 mm(4 μm) MAbPac RP column (Thermo Scientific, Waltham, Mass.) using mobile phase A and B containing 0.1% (v/v) formic acid in water, and 0.1% (v/v) formic acid in acetonitrile, respectively. A total of 0.6 μg sample was injected and the protein eluted using a linear gradient of 10 to 70% B over 7.0 minutes at a flow rate of 0.3 ml/min. Mass spectrometry detection using Synapt G2-Si was acquired in positive mode from m/z 100-2000 and the conditions were optimized as follows: sampling cone, 120 V; source offset, 100 V; source temperature, 90° C.; desolvation temperature, 120° C.; cone gas flow, 50 L/h; desolvation gas flow, 600 L/h. Calibration was performed using Glu-1-Fibrinopeptide B. Deconvolution of raw mass spectrum was performed with MaxEnt 3.1 (Waters) software

Peptide Mapping by Nano LC-MS/MS

Cell lysates were run by SDS gel electrophoresis and bands for GSDMD were excised, pooled and subjected to in-gel digestion with trypsin. The resulting peptides were lyophilized, re-suspended in 5% acetonitrile, 0.1% (v/v) formic acid in water and injected onto a NanoAcquity UPLC (Waters) coupled to a Q Exactive (Thermo Scientific) hybrid quadrapole orbitrap mass spectrometer. Peptides were trapped on a 100 μm I.D. fused-silica pre-column packed with 2 cm of 5 μm (200 Å) Magic C18AQ (Bruker-Michrom) particles in 5% acetonitrile, 0.1% (v/v) formic acid in water at 4.0 μl/min for 4.0 minutes. Peptides were then separated over a 75 μm I.D. gravity-pulled 25 cm long analytical column packed with 3 μm (100 Å) Magic C18AQ particles, at a flow rate of 300 nl/min containing mobile phase A, 0.1% (v/v) formic acid in water and mobile phase B, 0.1% (v/v) formic acid in acetonitrile, using a biphasic gradient: 0-60 min (5-35% B), 60-90 min (35-60% B), 90-93 min (60% B), 93-94 min (60-90% B), 94-109 (90% B), followed by equilibration to 5% B. Nano-ESI source was operated at 1.4 kV via liquid junction. Mass spectra were acquired over m z 300-1750 at 70,000 resolution (m z 200) with an AGC target of 1e6. Data-dependent acquisition (DDA) selected the top 10 most abundant precursor ions for tandem mass spectrometry by HCD fragmentation using an isolation width of 1.6 Da, max fill time of 110 ms, and AGC target of 1e5. Peptides were fragmented by a normalized collisional energy of 27, and product ion spectra were acquired at a resolution of 17,500 (m z 200). Raw data files were peak processed with Proteome Discoverer (version 2.1, Thermo Scientific) followed by identification using Mascot Server (Matrix Science) against the Mouse (Swissprot) FASTA file (downloaded July 2019). Search parameters included full tryptic enzyme specificity, and variable modifications of N-terminal protein acetylation, oxidized methionine, glutamine conversion to glutamic acid, 2-dimethyl succinylation, 2-monomethyl succinylation, 2-succination, and carbamidomethylation on cysteine. Assignments were made using a 10 ppm mass tolerance for the precursor and 0.05 Da mass tolerance for the fragment ions. All non-filtered search results were processed by Scaffold (version 4.8.4, Proteome Software, Portland, Oreg.) utilizing the Trans-Proteomic Pipeline (Institute for Systems Biology, Seattle, Wash.) at 1% false-discovery rate (FDR) for peptides and 99% threshold for proteins (2 peptides minimum).

Metabolite Extraction and Quantification

5×10⁷ cells in serum free medium were placed on ice and medium and cells were thoroughly scraped in 5 mL of 80% methanol solution and transferred to a 15 ml tube. Cells were then vortexed for 10 min at 4° C. Cell lysates were then centrifuged at 13,000 g for 10 min. Supernatant was collected and stored at −80° C. until processing for LC/MS. Samples were spiked with 2 μl of 7.5 μM ¹³C4 fumaric acid (Cambridge Isotope Laboratories, Tewksbury, Mass.) and dried in a SpeedVac. Proteins were precipitated with 200 μl of ice-cold 95% acetonitrile, 5% water (0.05% (v/v) formic acid), and the supernatant separated and dried by SpeedVac. The resulting residue was reconstituted in 50 μl of 95% acetonitrile, 5% water (0.05% formic acid) for liquid chromatography-tandem mass spectrometry (LC-MS/MS). For analysis, 5 μl aliquots were injected in technical triplicate onto a 2.1×150 mm (1.7 μm) BEH Amide (Waters) column using an Acquity UPLC (Waters) coupled to a Xevo TQ-XS (Waters) triple quadrupole mass spectrometer operating in the negative ion electrospray mode. The flow rate was 0.2 ml/min and mobile phase A (0.05% formic acid in water) and B (0.05% formic acid in acetonitrile) were used to elute fumarate using the following gradient conditions: 0-2 min (95% B), 2-8.2 min (95%-60% B), 8.2-8.5 min (60%-40% B), 8.5-9.5 min (40-95% B), and 9.5-13 min (95% B). Data was collected using selected reaction monitoring for fumarate and labeled internal standard using m z 114.9>71 and 119>74 mass transitions, respectively. A calibration curve was constructed in a lysate free solution of 95% acetonitrile, 0.05% formic acid in water. Two-fold serial dilutions were carried out using a working solution of 400 μM fumaric acid to obtain 12 points across the range of 0.08-400 μM, each spiked with 0.3 μM of ¹³C4 fumarate and were injected in technical duplicates. Peak areas were integrated in TargetLynx XS (Waters) and data analysis was carried out in Excel (Microsoft, Redmond, Wash.) and Prism (GraphPad Software, San Diego, Calif.). Sample concentrations were determined from the calibration curve (fitted with 1/X weight factor and R²=0.998). The relative standard deviations (RSDs) for the triplicate analyses were all within 10%.

Mass Spectrometry Data Analysis

Raw data files were peak processed with Proteome Discoverer (version 2.1, Thermo) followed by identification using Mascot Server (Matrix Science) against the Mouse (Swissprot) FASTA file (downloaded July 2019). Search parameters included trypsin enzyme, and variable modifications of N-terminal protein acetylation, oxidized methionine, glutamine conversion to glutamic acid, phosphorylation on serine and threonine, ubiquitination (GG on lysine), 2-dimethyl succinylation, 2-monomethyl succinylation, and 2-succinylation on cysteine. Assignments were made using a 10-ppm mass tolerance for the precursor and 0.05 Da mass tolerance for the product ions. All non-filtered search results were processed by Scaffold (version 4.8.4, Proteome Software, Inc.) utilizing the Trans-Proteomic Pipeline (Institute for Systems Biology) at 1% false-discovery rate (FDR) for peptides and 99% threshold for proteins (2 peptides minimum).

Targeted PRM analyses were carried out with cell lysate samples to validate modified cysteines detected in DDA analysis and also to detect any unmodified cysteines that were identified in GasderminD protein sample (incubated with the corresponding the small molecule ligand), but might have missed due to stochasticity of the DDA. Skyline (an open source software from proteome.gs.washington.edu/software/skyline) was used to generate precursor ions for PRM data acquisition and further data analysis. A dot product score >0.9 (similarity score comparing the PRM mass spectrum and library MS/MS data) was used to confirm the identified peptides.

siRNA Gene Knockdown

WT BMDMs were transfected with control non-targeting siRNA or NRF2 specific siRNA (50 nM) for 48 hours and assayed as indicated. siRNA 1 Thermofisher, 4390815/Assay ID: s70521, siRNA 2 Thermofisher 4390815/Assay ID: s70523.

LPS Septic Shock Model

Mice aged 8-12 weeks were administered LPS 5 mg/kg by intraperitoneal injection. Serum was collected after 5 h for ELISA analysis. For survival studies, mice were administered LPS 50 mg/kg intraperitoneally and monitored every 12 h for up to 96 h.

In Vitro Succination Reaction

5 μg of purified Human or Murine gasdermin-d was incubated with 50 μM of Fumarate or MMF in 50 μl of incubation buffer (250 mM HEPES, 2 mM EDTA and 0.1 mM neocuproine) and incubated for 3 hrs at 37 C.

In Vitro Binding Assay

HEK293T cells were transfected with 1 μg of Caspase 1 and immunoprecipitated on protein A/G beads. gasdermin-d was pre-succinated in vitro as above and incubated with Caspase1 protein A/G beads. Caspase1 beads were incubated with or without native recombinant gasdermin-d in 1 ml of lysis buffer rotating for 6 hrs at 4° C. overnight.

Induction and Assessment of EAE

Mycobaterium tuberculosis H37Ra (231141) was from BD. Incomplete Freund's adjuvant (F5506) was from Sigma-Aldrich. MOG35-55 peptide (residues 35-55, Met-Glu-Val-Gly-Trp-Tyr-Arg-Ser-Pro-Phe-Ser-Arg-Val-Val-His-Leu-Tyr-Arg-Asn-Gly-Lys (SEQ ID NO:1)) was synthesized by Sangon Biotech. EAE was induced using MOG35-55 peptide (200 μg per mouse) emulsified with CFA (50 μl per mouse, including 4 mg/ml M. tuberculosis H37Ra) and 50 μl of incomplete Freund's adjuvant. 250 ng of Pertussis toxin was administered IV on day 0 and 2 post immunization. Mice were scored daily for clinical signs of EAE in a blinded fashion. EAE score was calculated as follows: 0.5, partial tail paralysis; 1, tail paralysis; 1.5, reversible corrective reflex impairment; 2, corrective reflex impairment; 2.5, one hindlimb paralysis; 3, both hindlimbs paralysis; 3.5, both hindlimbs paralysis and one forelimb paralysis; 4, hindlimb and forelimb paralysis; and 5, death.

Histological and Immunohistochemistry

Tissue blocks were sectioned at 5 μm thick. For paraffin-embedded tissue, CNS tissue was rapidly dissected from mice perfused with PBS. Tissues were fixed in 4% paraformaldehyde overnight. Tissue sections were stained with H&E for evaluation of inflammation. IHC staining of GSDMD was performed with anti-GSDMD antibody (Abcam, ab219800) and detected using horseradish peroxidase-conjugated secondary antibodies after heat-induced antigen retrieval as previously described (18). Diaminobenzidine was used for detection. Images were captured with a Nikon Ds-Ri2 microscope. Histological scores: 0, no inflammatory cell infiltration and no demyelination; 1, slight inflammatory cell infiltration or demyelination observed; 2, moderate inflammatory cell infiltration or demyelination in several spots; 3, substantial inflammatory cell infiltration and large area of demyelination.

DMF Mouse Diet

DMF embedded mouse diet was formulated by Lab Diets®. DMF was added to a base diet of laboratory lab diet 5001 at a dose 100 mg/kg/day of consumption. Mice received the control base diet or the DMF containing diet for 6 weeks.

Human Brain Autopsies

Post-mortem lesions were collected from PMS patients (Table 2) as previously described (24). Briefly, tissue blocks fixed in 4% paraformaldehyde were 30 μM sections were fixed on superfrost slides and stained for GSDMD-N p30 (Cell signalling, 36425s) as previously described. IHC staining was performed by applied pathology systems, Shrewsbury, Mass.

Synthesis of methyl (E)-4-oxo-4-(propynylamino)-2-butenoate (MMF-alkyne, 1)

Propargylamine (0.141 mL, 2.2 mmol) and DIPEA (0.38 mL, 2.2 mmol) were added to a solution of mono-methyl fumarate (0.25 g, 1.9 mmol) and HATU (0.76 g, 2.0 mmol) in Dimethyl Formamide (5 mL) at ice cold condition. The reaction mixture was stirred at room temperature for 20 h. The reaction mixture was poured into water to precipitate the crude compound. This crude mixture was dissolved in CH₃CN:H₂O (1:4) and purified by preparative HPLC (CH₃OH:CH₃CN) to yield the product 1 as a white solid (90%). ¹H NMR (500 MHz, DMSO) δ 9.1 (bs, 1H), 7.0 (d, J=16.0 Hz, 1H), 6.7 (d, J=16.0 Hz, 1H), 4.16 (dd, J=5.3, 2.6 Hz, 2H), 3.80 (s, 3H), 3.2 (t, J=2.6 Hz, 1H). ¹³C NMR (DMSO) δ 166.08, 163.29, 135.76, 130.90, 78.80, 72.39, 52.45, 29.77. MS (ESI+): 168.2 (M+H⁺). ¹H and ¹³C NMR spectra were recorded in d₆-DMSO as solvent using a Bruker 500 MHz NMR spectrometer. Chemical shift values are cited with respect to TMS as the internal standard. Reverse-phase HPLC using a semi-preparative C18 column (Agilent, 21.2×250 mm, 10 μm) and a water/acetonitrile gradient supplemented with 0.05% trifluoroacetic acid.

Copper Click Chemistry

Cells treated with or without the MMF-Alkyne probe were lysed and quantified by protein DC assay. Proteome samples (2 mg/ml) were incubated with TCEP, TBTA ligand, copper sulphate and Biotin Azide for 1 hour at room temperature with vortexing every 15 minutes. Precipitated proteins were centrifuged for 5 minutes at 4,600 g. Protein pellets were washed twice with ice cold methanol and sonicated in 1.2% SDS. Samples were heated at 95° C. for 5 minutes and diluted to a final volume of δ ml with PBS (0.2% SDS). An aliquot of the post-clicked lysate was retained and the remainder was incubated with streptavidin beads on a rotator overnight at 4 C. Samples were rotated at room temperature for 2 hours to resolubilize the SDS. Beads were washed five times with 0.2% SDS/PBS and placed on a rotator for 10 minutes in between washes. Beads were washed with ultra-pure water a further 3 times. At this point beads were eluted in 1× sample buffer or further processed for mass spec as described.

PBMC Isolation

PBMC were isolated from whole blood of consenting donors. Blood was diluted 1:1 in sterile PBS and layered over 15 mls of Lymphoprep. Blood was spun at 2,000 RRM with no break. The interphase was transferred to a fresh tube using a Pasteur pipette and washed twice in PBS. Red blood cells were lysed in RBC lysis buffer for 10 mins at room temperature. Cells were washed once more in PBS and counted.

Ethics

All animal studies were performed in compliance with the federal regulations set forth in the Animal Welfare Act (AWA), the recommendations in the Guide for the Care and Use of Laboratory Animals of the National Institutes of Health, and the guidelines of the UMass Medical School Institutional Animal Use and Care Committee. All protocols used in this study were approved by the Institutional Animal Care and Use Committee at the UMass Medical School (protocols A-1633).

Statistical Analysis

For comparisons of two groups two-tailed students' t test was performed. Multiple comparison analysis was performed using two-way ANOVA. Mann-Whitney U test was used for EAE analysis. Mantel-Cox were used for survival analysis. Three to ten mice were used per experiment, sufficient to calculate statistical significance, and in line with similar studies published in the literature.

### Example 1. Succination Inactivates Gasdermin D and Blocks Pyroptosis

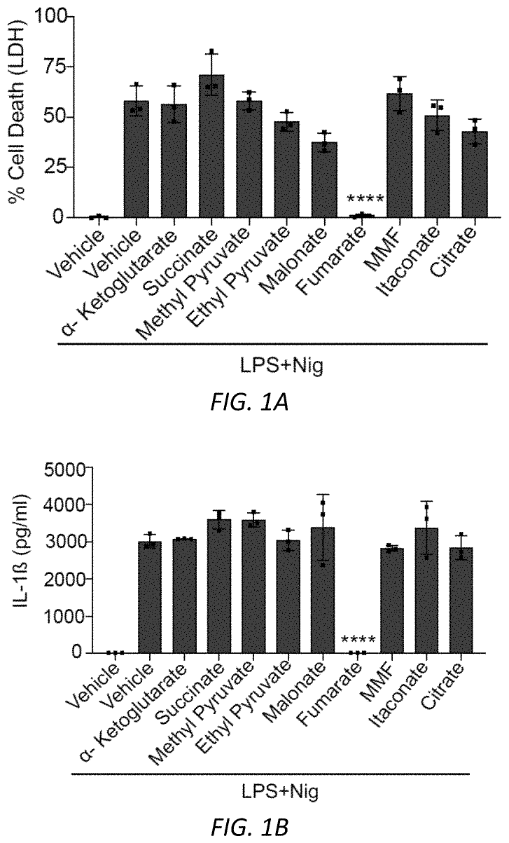

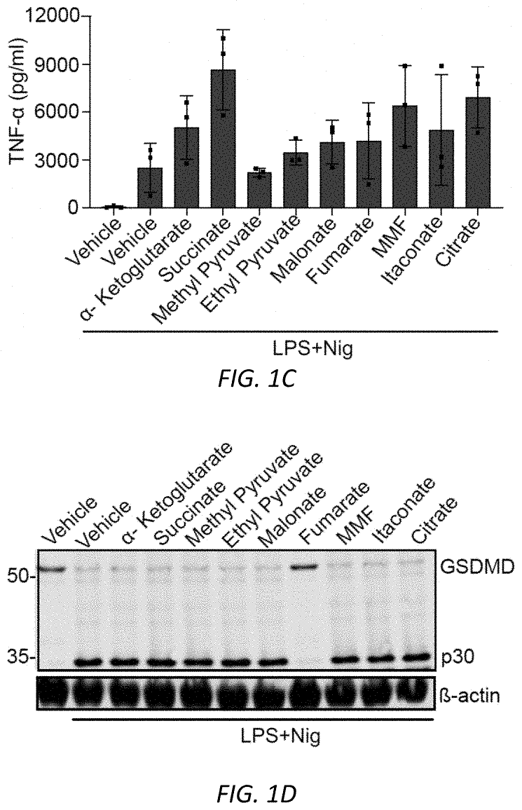

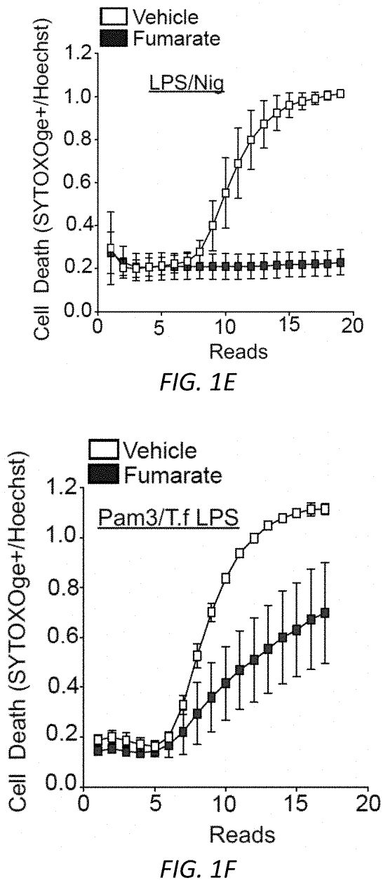

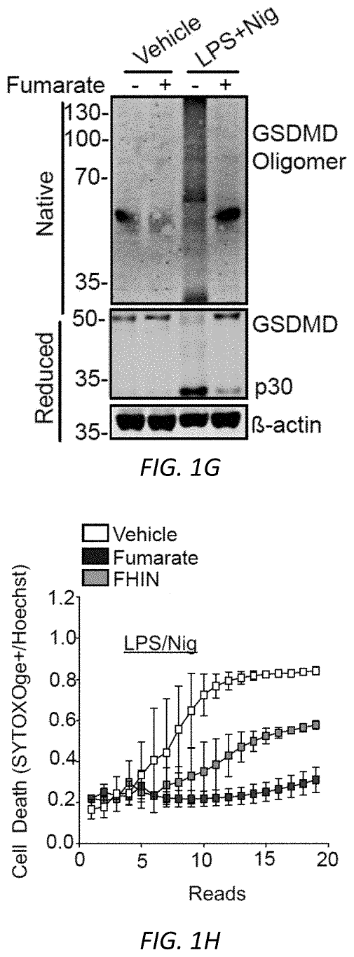

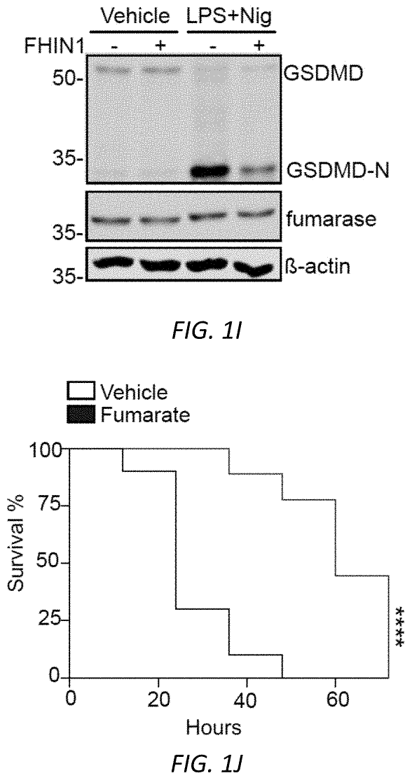

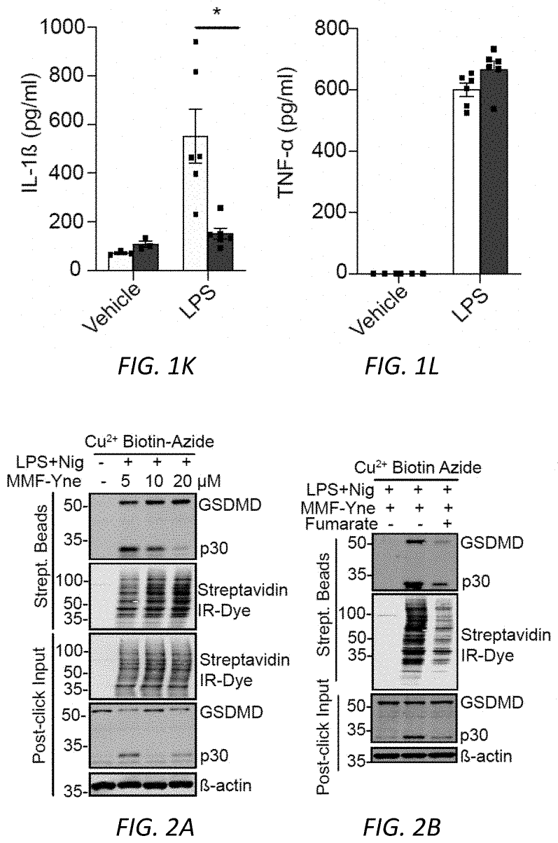

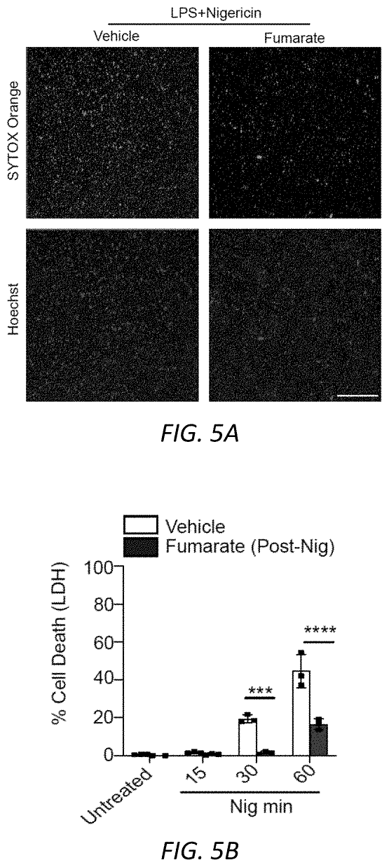

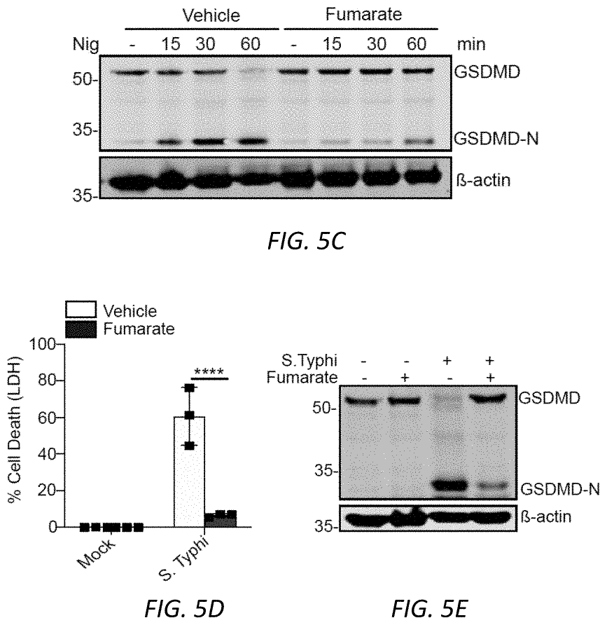

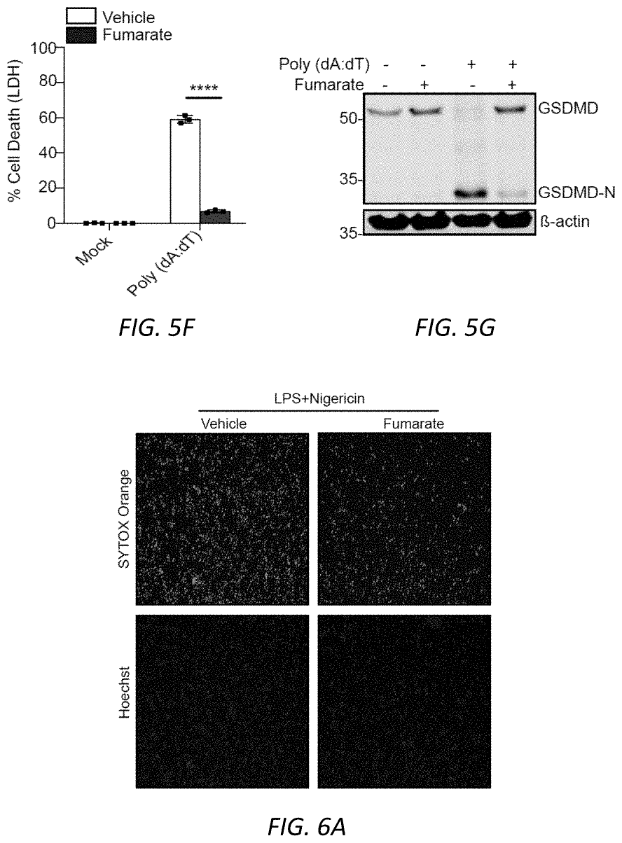

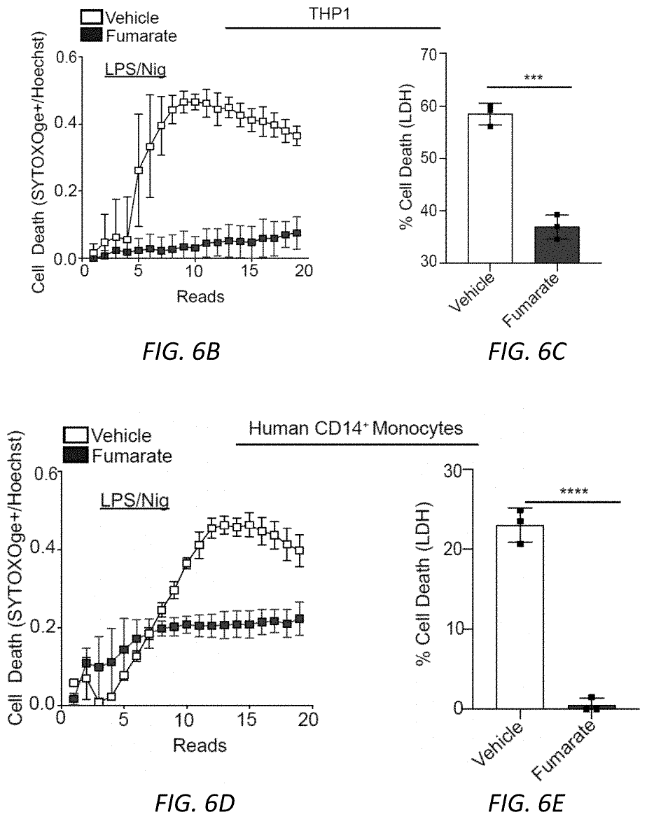

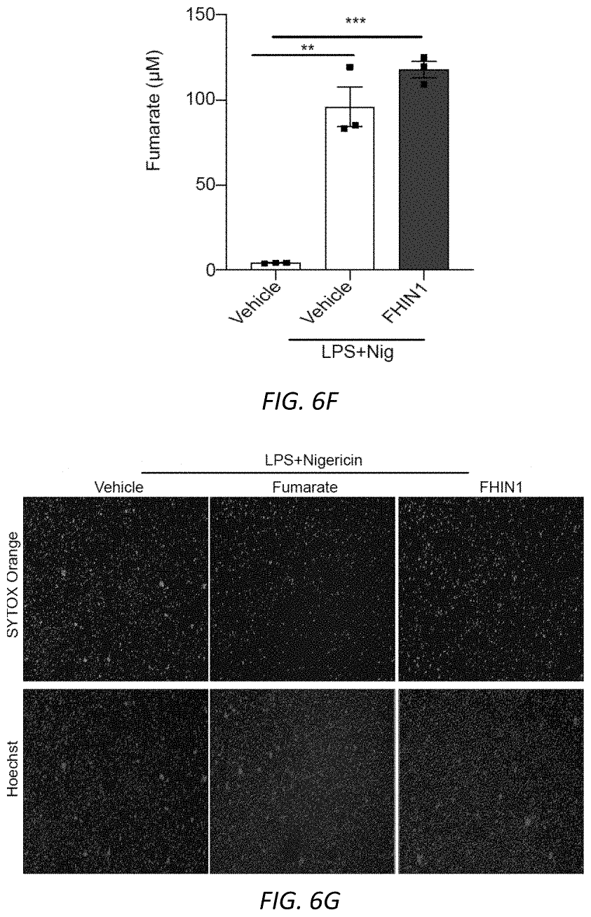

Numerous studies have shown that cell metabolism impacts inflammatory responses. LPS activated macrophages switch from oxidative phosphorylation to aerobic glycolysis. Krebs cycle intermediates such as succinate and itaconate accumulate and moonlight as positive and negative regulators of inflammatory gene expression (3-8). To determine if Krebs cycle intermediates modulate pyroptosis, we tested their effect on inflammasome responses. Bone marrow-derived macrophages (BMDMs) were primed with LPS (2 hr) before being exposed to metabolites and then exposed to nigericin (Nig) and inflammasome activation/pyroptosis measured. The metabolites were added after LPS to avoid any impact on transcription. Dimethyl fumarate, hereafter called fumarate, potently blocked LPS-Nig-induced release of LDH (FIG. 1A) and IL-113 (FIG. 1B) but not TNF-α (FIG. 1C). Fumarate also blocked formation of GSDMD-N (FIG. 1D). Monomethyl fumarate (MMF), a cell impermeable derivative of fumarate, displayed no inhibitory effect. Fumarate impaired kinetic cell death (0-6 hrs) (FIG. 1E, FIG. 5A). Fumarate even impaired cell death when added after Nig treatment (FIG. 5B, C). The inhibitory effect of fumarate was also observed in BMDMs after transfected LPS (FIG. 1F), Salmonella infection (FIG. 5D-E) or poly (dA:dT) (FIG. 5F-G). Fumarate also blocked LPS-Nig induced cell death and LDH release in human THP1 (FIG. 6A-C) and CD14⁺ monocytes (FIG. 6D-E). BMDMs pre-treated with fumarate had decreased GSDMD oligomerization in response to LPS-Nig, indicating that fumarate blocks pore formation (FIG. 1G).

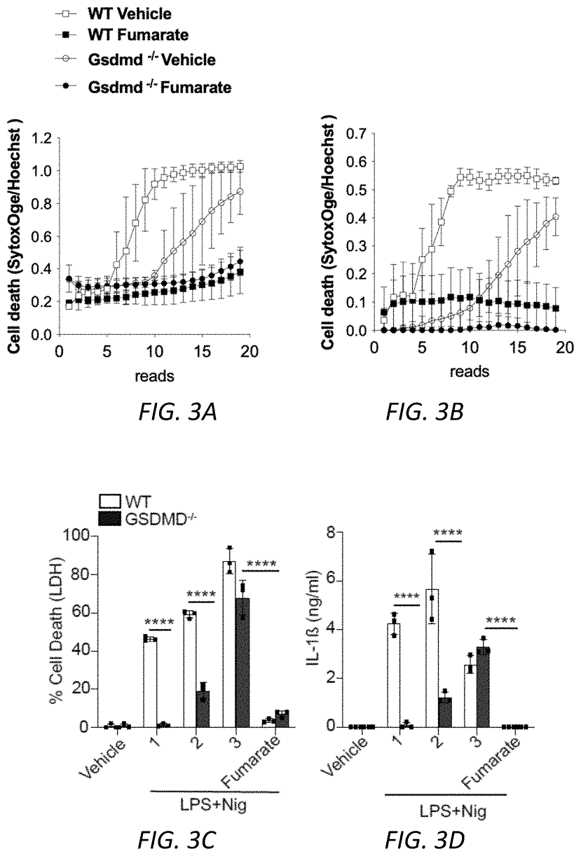

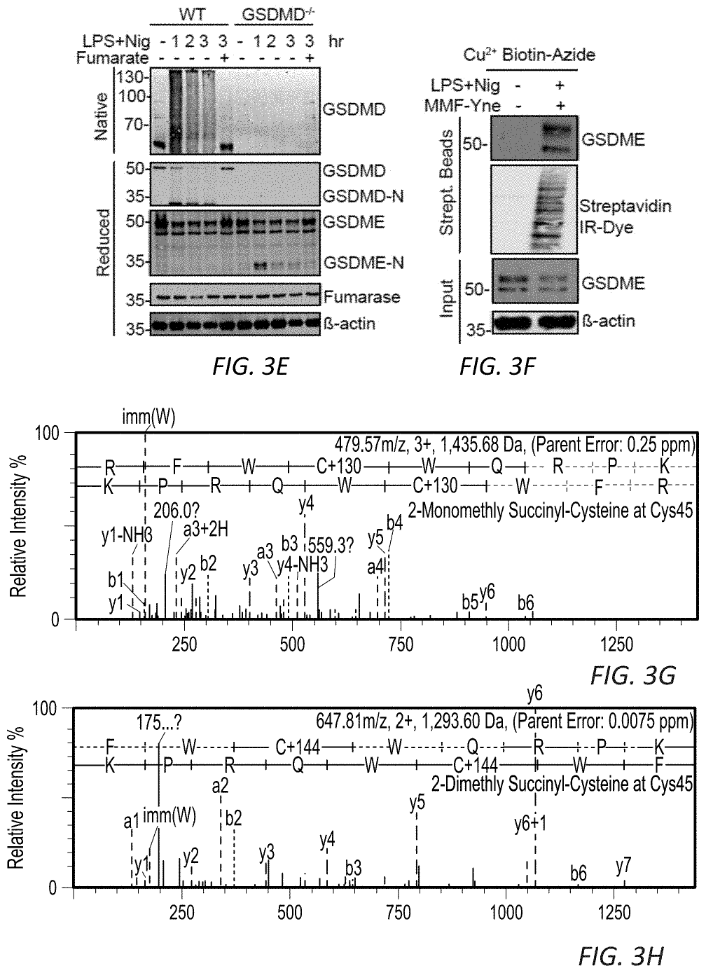

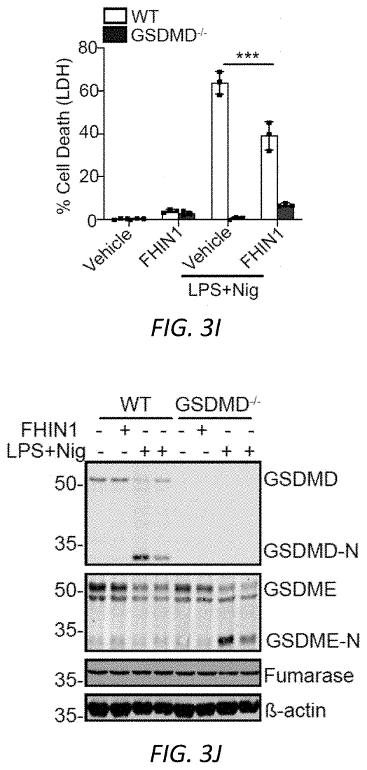

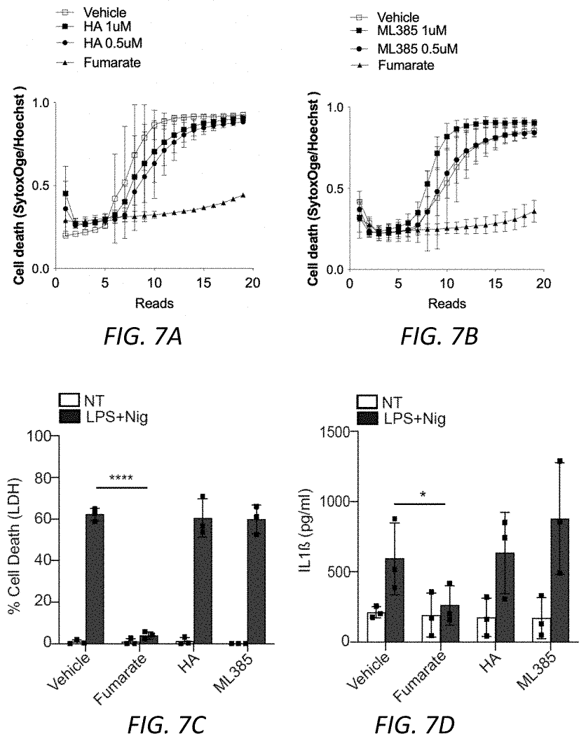

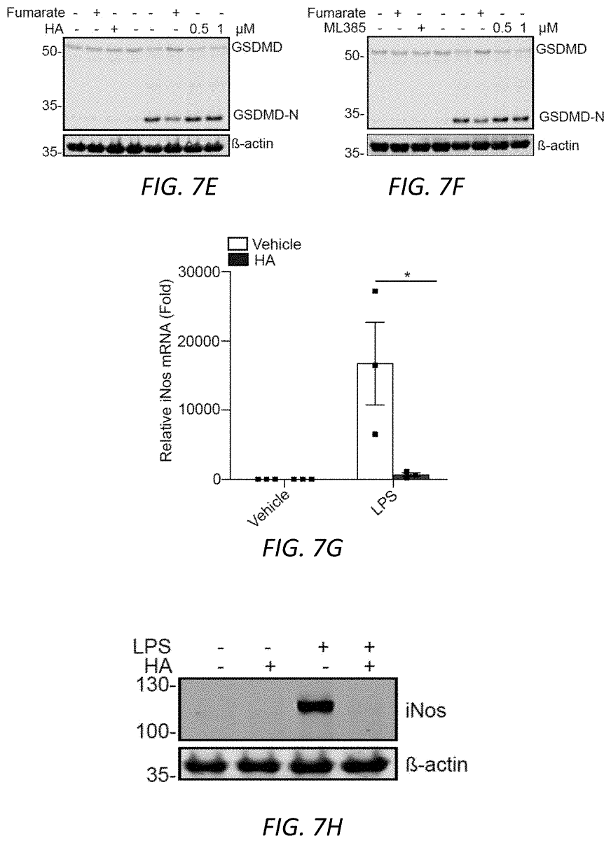

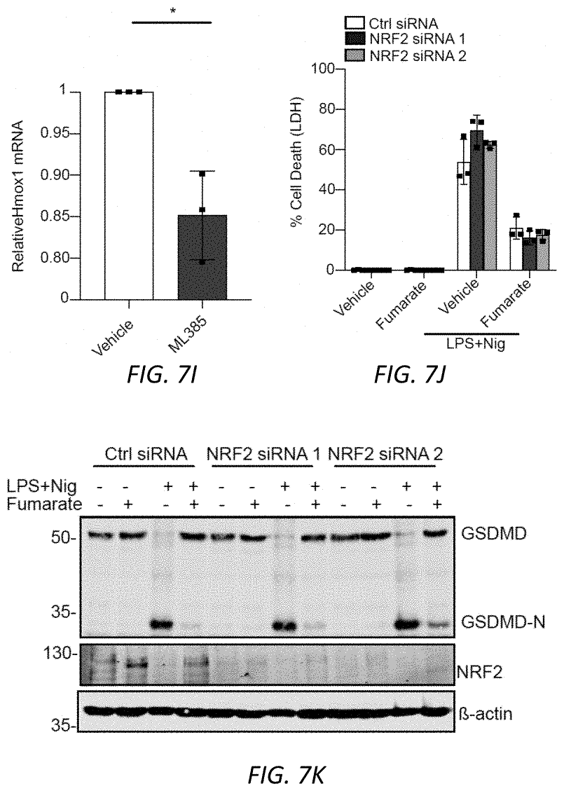

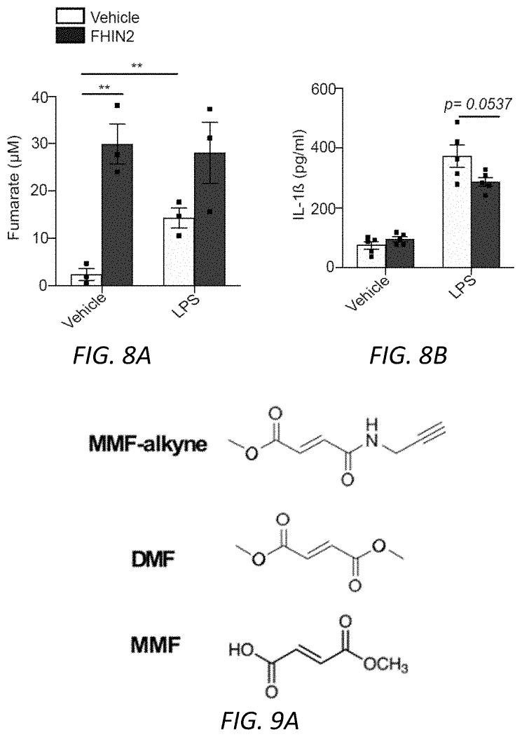

Fumarate accumulates in LPS activated macrophages (FIG. 6F), similar to prior studies (5, 7). To assess the effect of endogenous fumarate, we blocked fumarate hydratase using FHIN1, allowing further increases in cells (FIG. 6F). FHIN1 impaired cell death (FIG. 1H, FIG. 6G), and reduced the formation of GSDMD-N (FIG. 1I). Fumarate has previously been reported to exhibit anti-inflammatory activity through NRF2 or GAPDH (9, 10). Cells treated with the GAPDH inhibitor Heptilidic acid (HA) (11) or the NRF2 inhibitor ML385 (12) had no impact on cell death (FIG. 7A-B), LDH release (FIG. 7C), IL-1β release (FIG. 7D) or GSDMD-N formation (FIG. 7E-F). HA and ML385 inhibited the GAPDH and NRF2 target genes iNos and Hmox1 (FIG. 3G-I). siRNA knockdown of NRF2 also failed to impact pyroptosis (FIG. 7J-K). Thus, the regulatory effects of fumarate are independent of NRF2 or GAPDH. Fumarate also suppressed pyroptosis in vivo. WT mice receiving a lethal dose of LPS succumbed to LPS septic shock within 48 hours whereas mice administered LPS and a single dose of fumarate had increased survival (FIG. 1J). Fumarate reduced IL-1β (FIG. 1K) but not TNF-α levels (FIG. 1L). An in vivo compatible fumarate hydratase inhibitor, FHIN2, which elevated fumarate levels in vivo also reduced IL-1β levels (FIG. 8A-B).

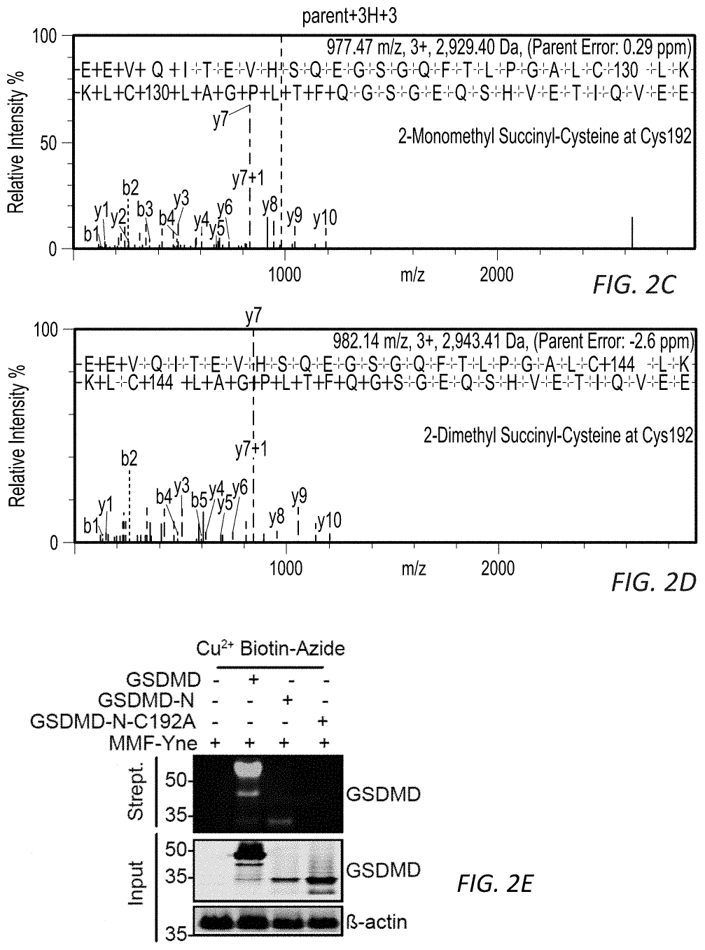

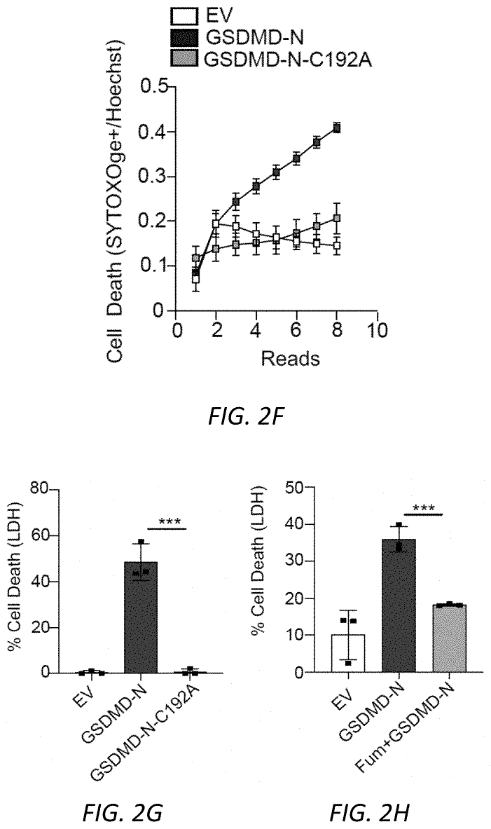

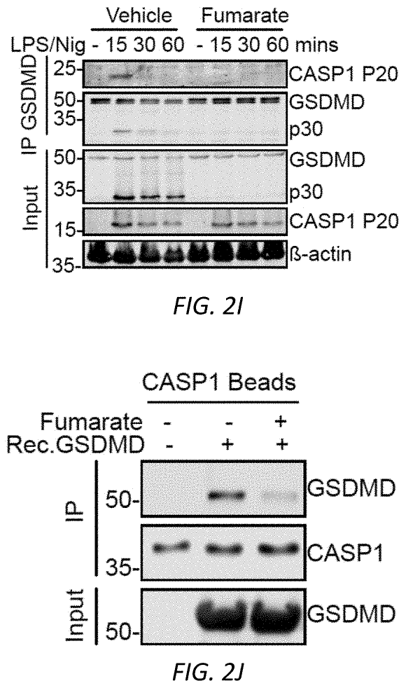

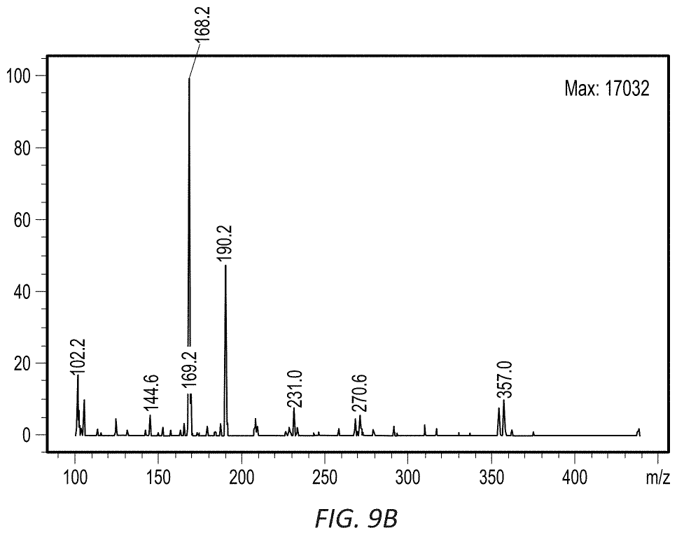

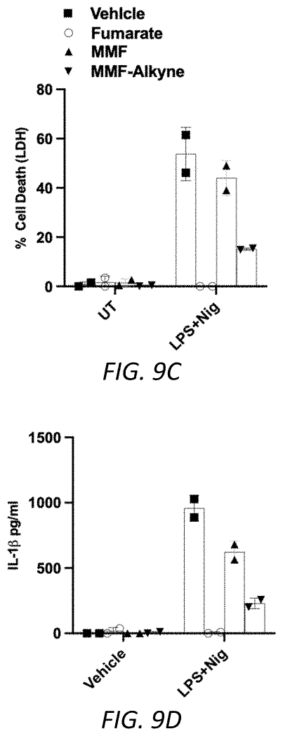

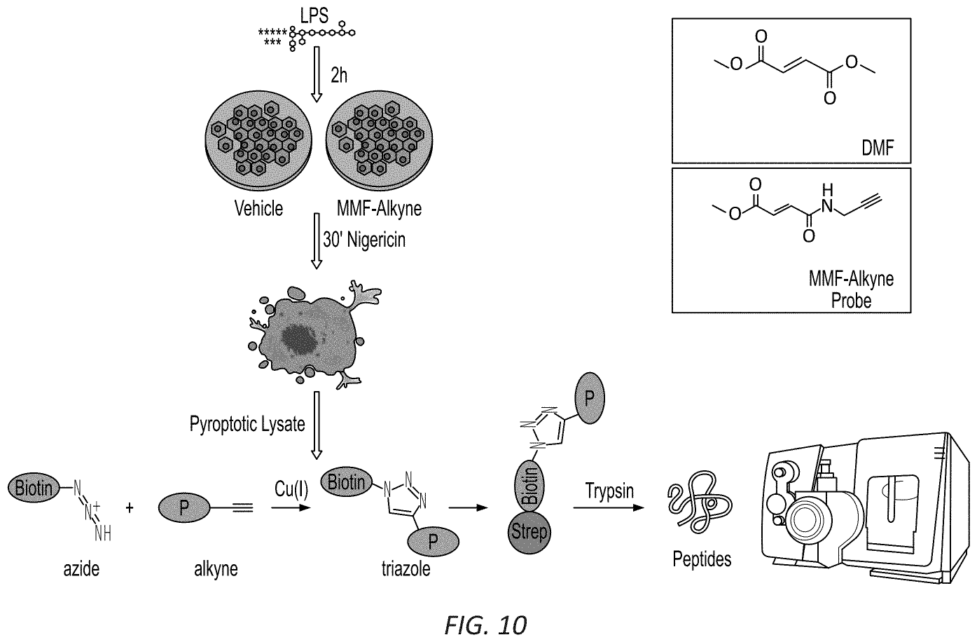

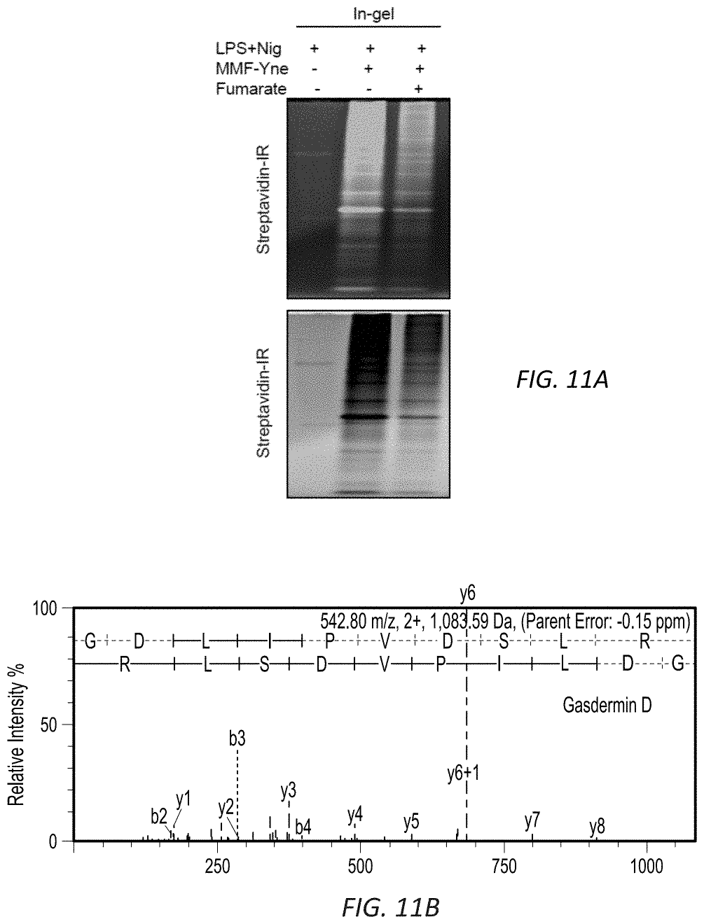

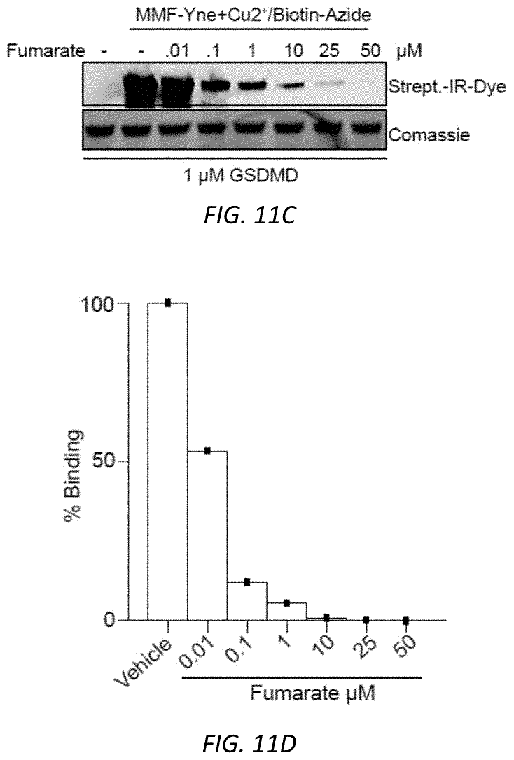

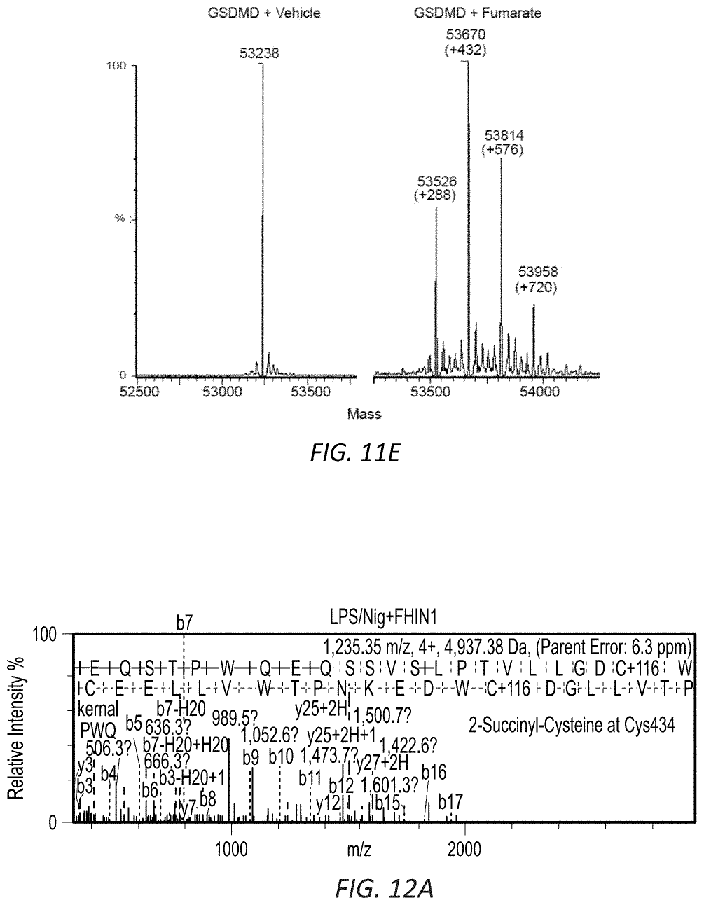

We next employed a chemoproteomic approach to identify targets of fumarate. We synthesized monomethyl-fumarate alkyne (MMF-Yne), a cell-permeable fumaric acid-alkyne (13) which mimics fumarate but has an alkyne handle for target identification (FIG. 9A-B). Like fumarate, MMF-Yne inhibited LPS-Nig-induced LDH release (FIG. 9C) and IL-1β (FIG. 9D). To identify MMF-Yne-bound targets, we employed click chemistry (FIG. 10). Immunoblotting revealed that the probe reacts with multiple proteins in pyroptotic lysates (FIG. 11A). Treatment with unlabeled fumarate reduced the MMF-Yne signal (FIG. 11A). Mass spectrometry identified GSDMD as an MMF-Yne target (FIG. 11B). Notably, MMF-Yne dose-dependently labeled GSDMD (FIG. 2A). Furthermore, fumarate blocked MMF-Yne labeling of GSDMD (FIG. 2B). Maximum occupancy of 1 μM GSDMD was achieved at 25 μM fumarate (FIG. 11C-E).

Fumarate derivatizes protein cysteines to generate 2-(succinyl)-cysteine, an irreversible post translational modification that impacts protein function (9). LCMS/MS peptide mapping experiments showed that treatment of recombinant human or murine GSDMD with MMF led to abundant monomethyl succination (2-monomethyl succinyl-cysteine) at Cys¹⁹¹ in Human and Cys¹⁹² in mouse GSDMD. Fumarate also modified (2-dimethyl succinyl-cysteine) GSDMD at the same cysteines. Neither of these were detected in vehicle controls. In addition to Cys¹⁹², murine GSDMD was succinated on nine other cysteines (table 1). Human GSDMD was succinated on four additional cysteines (table 1). We also immunoprecipitated GSDMD from fumarate treated BMDMs and analyzed tryptic digests by tandem mass spectrometry. This approach revealed a combination of 2-monomethyl and 2-dimethyl succination of GSDMD on Cys¹⁹² (FIG. 2C-D) as well as Cys⁵⁷ and Cys⁷⁷. LPS-Nig treatment in the presence of FHIN1 also modifies GSDMD (FIG. 12A-B) Thus, GSDMD is succinated by exogenous or endogenous fumarate.

TABLE 1
In vitro cysteine succination sites in GSDMD and GSDME

| Species | | |
|---|---|---|
| h.GSDMD | m.GSDMD | h.GSDME |

| MMF | DMF | MMF | DMF | MMF | DMF |
|---|---|---|---|---|---|
| Cys56 | Cys56 | Cys39 | Cys39 | Cys45 | Cys45 |
| Cys191 | Cys191 | Cys57 | Cys57 | Cys156 | Cys156 |
| Cys268 | Cys268 | Cys77 | Cys77 | Cys168 | Cys180 |
| Cys309 | Cys309 | Cys192 | Cys122 | Cys180 | Cys235 |
| Cys467 | Cys467 | Cys265 | Cys192 | Cys235 | Cys371 |
| | | Cys299 | Cys265 | Cys371 | Cys408 |
| | | Cys434 | Cys299 | Cys417 | Cys489 |
| | | Cys487 | Cys434 | Cys489 | |
| | | | Cys448 | | |
| | | | Cys489 | | |

Cys¹⁹² (Cys¹⁹¹ in humans) is critical for GSDMD-N oligomerization (14). MMF-Yne modifies full length and GSDMD-N but not GSDMD-N-C192A (FIG. 2E). While GSDMD-N induces cell permeability and LDH release in HEK293T cells, consistent with previous studies (14, 15) GSDMD-N-C192A did not (FIG. 2F-G). Fumarate inhibited the GSDMD-N induced LDH release (FIG. 2H). Since fumarate can impair both processing and activity of GSDMD, we hypothesized that succination may prevent caspase 1-GSDMD interactions. Indeed, fumarate completely blocked this (FIG. 2I). Importantly, processing of caspase 1 was not impaired by fumarate. Succination of GSDMD in vitro also reduced its binding to caspase 1 immunoprecipitated from cells (FIG. 2J). Thus, fumarate modifies GSDMD blocking its processing, oligomerization, and cell death.

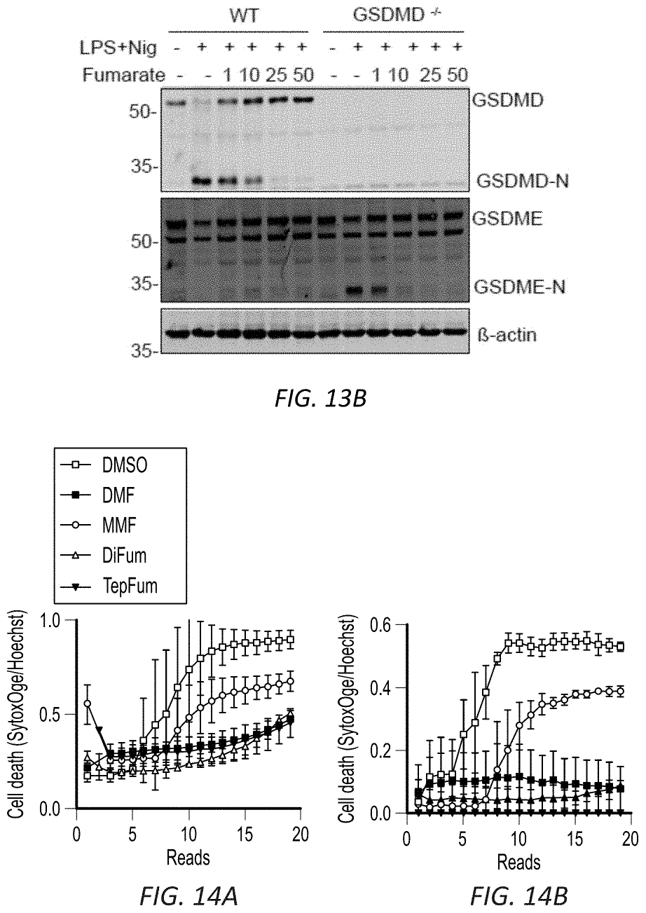

GSDMD is critical for pyroptosis, however in its absence cell death still occurs albeit with slower kinetics (2). Consistently, fumarate inhibited cell death in both WT and GSDMD-deficient BMDMs (FIG. 3A) and THP1 cells (FIG. 3B). Similar findings were observed when LDH (FIG. 3C) and IL-1β levels were measured (FIG. 3D). Gasdermin E (GSDME) drives apoptosis in GSDMD-deficient cells (16). GSDME processing in GSDMD-deficient cells was also blocked by fumarate (FIG. 3E). Fumarate inhibited GSDMD and GSDME-driven cell death comparably (FIG. 13A-B). MMF-Yne also modifies GSDME (FIG. 3F). Indeed, fumarate and MMF succinated GSDME at Cys⁴⁵ (FIG. 3G-H) and additional sites (table 1). Treatment of cells with FHIN1 attenuated GSDMD-independent (GSDME-dependent) cell death (FIG. 31) and the generation of GSDME-N (FIG. 3J). Thus, fumarate modifies GSDMD and GSDME by succination.

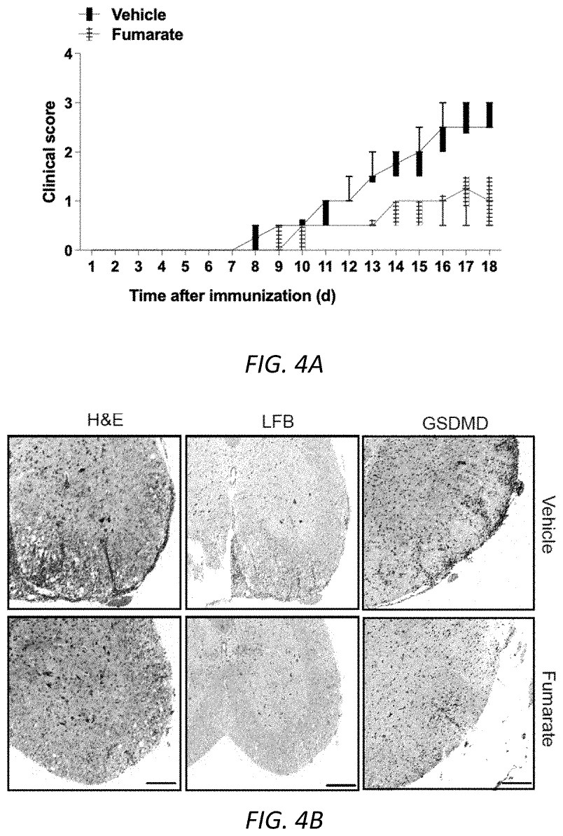

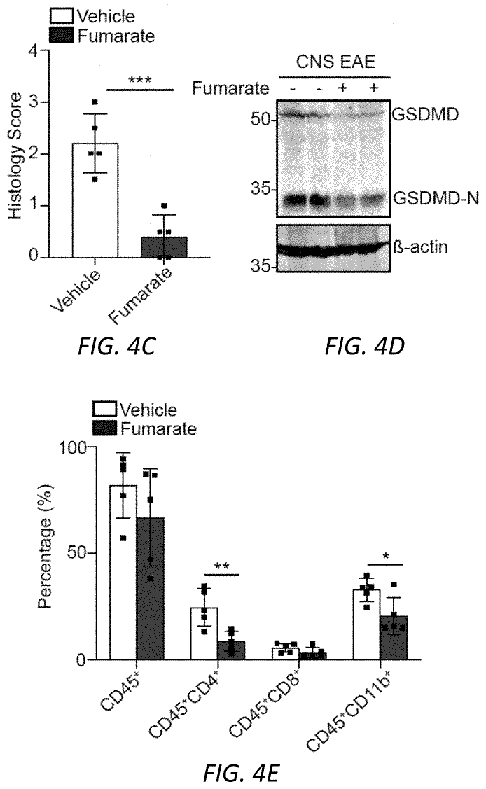

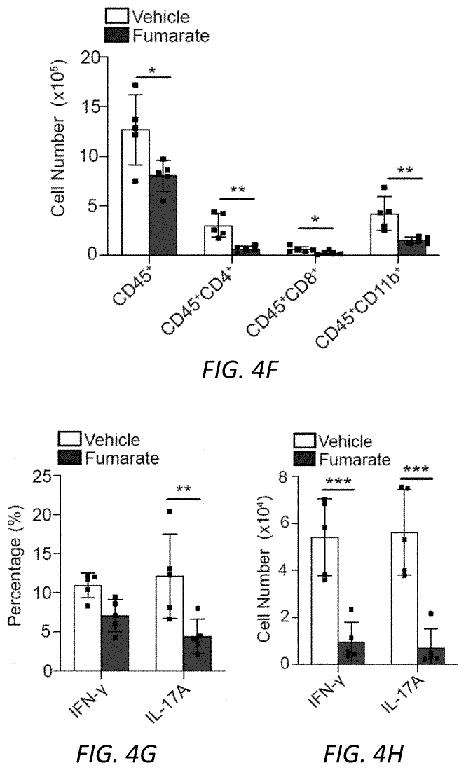

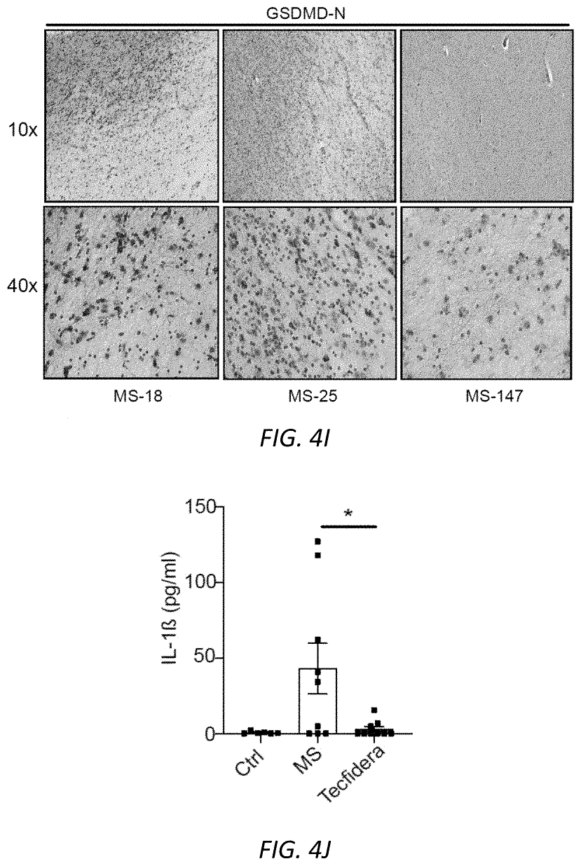

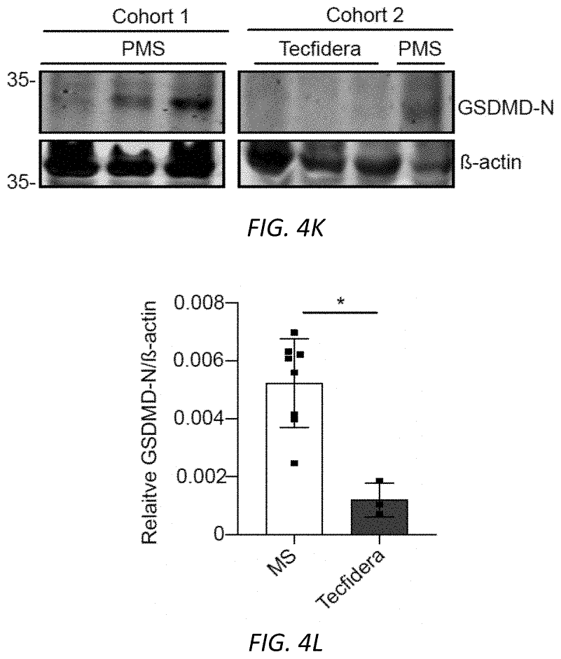

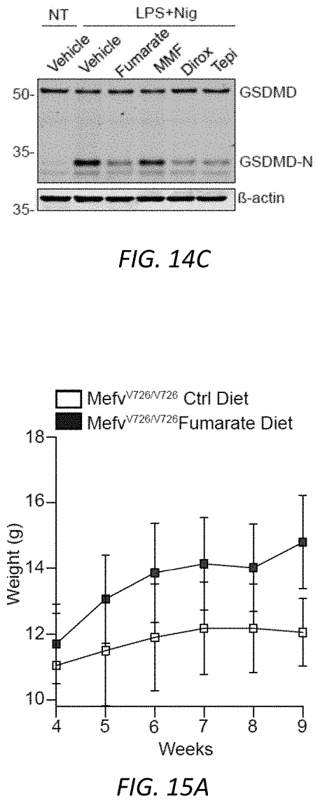

GSDMD is an important driver of inflammatory diseases (17). GSDMD-deficient mice are protected from experimental autoimmune encephalomyelitis (EAE) (18). Notably, fumarate analogues such as DMF are FDA-approved for the treatment of Multiple Sclerosis (MS). Two newer MS drugs, diroximel fumarate and tepilamide fumarate also blocked LPS-Nig induced pyroptosis and GSDMD-N formation (FIG. 14A-C). Fumarate blocked the onset of EAE, reduced neuropathology and demyelination (FIG. 4A-C). Fumarate also reduced GSDMD-N in CNS tissue (FIG. 4B, D). GSDMD is essential for cell infiltration to the CNS during EAE. Mice receiving fumarate had reduced infiltration of myeloid cells, CD4⁺ and CD8⁺ T-cells (FIG. 4E-F). Fumarate reduced Th1 (IFN-γ⁺) and Th17 (IL-17A⁺) cells in the CNS (FIG. 4G-H). Post-mortem brain tissue from MS patients stained positive for GSDMD-N (FIG. 4I, Table 2).

TABLE 2
Clinical evaluation patient information related to FIG. 41.

| Patient # | Gender | Age | MS Type | EDSS | PMI (h) |
|---|---|---|---|---|---|
| MS-18 | Male | 45 | SPMS | 7 | 3 |
| MS-25 | Male | 56 | SPMS | 9.5 | 3 |
| MS-147 | Female | 50 | RR | 3 | 7 |

Expanded disability status scale (EDSS), post-mortem interval hours (PMI). Secondary progressive MS (SPMS). Relapsing remitting (RR).

Patients with MS had elevated levels of IL-1β and GSDMD-N in PBMCs (FIG. 4J-L, Table 3). Both IL-1β and GSDMD-N were reduced in patients taking fumarate (Tecfidera) (FIG. 4J-L, Table 3). Together, these findings indicate that fumarate reduced GSDMD-driven responses in EAE and support a model where elevated GSDMD contributes to MS.

TABLE 3
Clinical evaluation and patient information related to FIG. 4I-L.

| Patient # | Gender | Age | BMI | Diagnosis | MS Type | EDSS | SDMT | 25 Ft-walk | Peg Test | Tecfidera |
|---|---|---|---|---|---|---|---|---|---|---|
| 21 | Male | 62 | 26.18 | 1985 | RRPMS | 6.5 | 54 | 9.09 | 25 | − |
| 22 | Female | 50 | 31.68 | 1992 | RPMS | 6.5 | 38 | 25.32 | 36.41 | − |
| 93 | Female | 58 | 21.28 | N/A | SPMS | 6.5 | 57 | 20.6 | 13.2 | − |
| 321 | Male | 73 | 24.64 | 1989 | PMS | 6.5 | 50 | 8.6 | 17.5 | − |
| 137 | Female | 53 | 22.25 | 2004 | SPMS | 6.5 | 43 | 6.8 | 10.5 | − |
| 324 | Female | 52 | 21.22 | 1994 | SPMS | 5 | 32 | 8 | 15.35 | − |
| 326 | Male | 65 | 31.71 | N/A | SPMS | 8 | 22 | N/A | 38.7 | − |
| 328 | Female | 61 | 39.74 | N/A | SPMS | 8.5 | N/A | N/A | N/A | − |
| 132 | Female | 51 | 19.97 | 1991 | SPMS | 6.5 | 37 | 13.13 | 78 | − |
| 337 | Female | 41 | 21.1 | 2009 | RR | 1.5 | 65 | 3.3 | 10.7 | + |
| 253 | Female | 58 | 23.53 | 1997 | SPMS | 4.5 | 21 | 17.53 | 17.53 | + |
| 301 | Male | 68 | 27.28 | 2005 | PMS | 7 | 31 | 22.1 | 22.1 | + |
| 224 | Female | 73 | 26.26 | 1985 | PMS | 6 | 21 | 25.5 | 25.5 | + |
| 320 | Female | 67 | 30.36 | 1987 | SPMS | 3.5 | 33 | 10.3 | 10.3 | + |
| 218 | Female | 53 | 18.36 | 2018 | SPMS | 1.5 | 58 | 10.22 | 10.22 | + |
| 253 | Female | 59 | 23.53 | 1996 | SPMS | 4.5 | 21 | 17.53 | 17.53 | + |
| 342 | Female | 58 | 30.07 | 1984 | SPMS | 1 | 50 | 12.59 | 12.59 | + |
| 257 | Male | 44 | 36.1 | 2018 | RR | 3.5 | 43 | 5.9 | 19.8 | + |

Expanded disability status scale (EDSS), Symbol digit modalities test (SDMT), times 25-foot walk test (25 Ft-walk), 9-hole peg test (Peg Test), body mass index (BMI), Not Available (N/A). Refractory relapsing PMS, (RRPMS), progressive MS (PMS), secondary progressive MS (SPMS), relapsing remitting (RR).

### Example 2. Fumarate-Induced Succination of Gasdermin D for the Treatment of ARDS During COVID-19 Infection

SARS-CoV2 is a recently emerged virus that has led to a global pandemic. As of April 2020, there are over 3 million cases worldwide and ˜230,000 deaths reported. Coronaviruses are a family of RNA viruses found in humans and other animals that can result in the development of acute and chronic diseases of the upper and lower respiratory tract. To date 7-coronavirus species are known to cause human diseases. Four species typically result in common cold symptoms and two, SARS-CoV and Middle East respiratory syndrome coronavirus (MERS-CoV), cause severe respiratory disease. SARS-COV2 results in a disease termed COVID-19. Through analysis of global data, it has been established that the respiratory symptoms of COVID-19 are unpredictable and heterogeneous, with infections ranging from asymptomatic to significant hypoxia with Acute Respiratory Distress Syndrome (ARDS)(26). Indeed, in severe cases of SARS-CoV2 infection ARDS is a primary cause of mortality. Multiple studies have identified increases in inflammatory cytokines such as IL-6, TNF, IL-1b and IL-18(27-29). In addition, patients with severe ARDS display increased production of Lactate dehydrogenase (LDH)(30). This “cytokine storm” is typically associated with pulmonary damage, respiratory distress, and unfavorable outcomes. Whilst the development of a vaccine is 12-18 months away from widespread circulation, existing therapeutics are now being assessed for anti-viral or anti-inflammatory properties to alleviate and treat more severe forms of COVID-19 infection. Identifying, existing safe FDA-approved therapies will reduce the mortality associated with infection and reduce the overall demand for patient care. Numerous studies have highlighted the importance of inflammasomes as key mediators of ARDS (31). Inflammasomes are multimeric complexes that control the release of the pro-inflammatory cytokines IL-1β and IL-18 through a form of cell death termed pyroptosis (1, 25-33). Recently, a pore forming protein Gasdermin D (GsdmD) was identified as the executioner of Pyroptosis(34, 35). Inactive in resting cells, GsdmD is cleaved by Caspase 1 during inflammasome activation leading to the release of an active N-terminal pore-forming fragment. Following cleavage GsdmD is ubiquitinated, undergoes oligomerization and inserts into the plasma membrane resulting in pore formation, loss of membrane integrity and release of cytokines (2) Recently, GsdmD has been identified as a key driver in the pathogenesis of numerous inflammatory diseases. Dimethyl Fumarate (DMF), trade name Tecfidera®, is a frontline treatment for Multiple Sclerosis. DMF is an ester derivative of the TCA-cycle metabolite Fumarate (36). Mechanistically, DMF and its therapeutically relevant metabolite Monomethyl Fumarate (MMF) modify cysteine residues by a reaction known as Succination (15). Indeed, inactivation of GAPDH dependent pro-inflammatory transcriptional responses by succination has been proposed as one mechanism of action for DMF in MS patients (16). However, the therapeutic anti-inflammatory effects of DMF cannot be entirely explained by inactivation of GAPDH. Exciting new data from our group has identified GsdmD as a target of DMF. Using a combination of innovative In vitro and cell-based methods we have shown that DMF and MMF succinate GasderminD. Succination of GasderminD blocks its cleavage, ubiquitination, oligomerization, pyroptosis and release of inflammatory cytokines. Thus succination of GasderminD by DMF may antagonize pyroptotic cell death and a inflammation and alleviate ARDS in COVID19 patients. We believe DMF could be repurposed and used as a frontline therapy for treating COVID-19 associated morbidities.

### Example 2.1: Analysis of GsdmD Activity and Pyroptotic Cell Death in COVID-19 ARDS Patients

We determine the activation state of GsdmD in patients that have COVID-19. Consenting patients as well as healthy controls donate blood for the proposed experiments. GsdmD cleavage, ubiquitination, and oligomerization are measured in Peripheral Blood Mononuclear Cells (PBMCs) from patients and healthy controls. Analysis of GsdmD activation states is carried out using established biochemical techniques from our lab. Following activation GsdmD is processed by CaspaseI into an active N-terminal p30 fragment. The p30 fragment then oligomerizes and forms membrane pores. Native and reduced protein fractions from patient PBMCs lysates are compared for processing and oligomerization using western blotting. As shown above, we have also determined that ubiquitination of GsdmD is an essential prerequisite for activity. To measure GsdmD ubiquitination Tandem Ubiquitin Binding Entities (TUBEs) agarose beads are used to enrich for ubiquitinated proteins in patient PBMC lysates. Bead pulldowns are blotted for GsdmD to determine the extent of GsdmD ubiquitination in COVID-19 patients. IL-1β and IL-18 levels are also measured in patient plasma collected from the corresponding patients. The levels of IL-1β and IL-18 are correlated with GsdmD activation. The proposed experiments determine the activation state of GsdmD in patients with both mild and more severe COVID-19 states. All patient data are correlated with viral load which will be measured by serial swabs of nasopharyngeal passages.

### Example 2.2: Ex Vivo Treatment of Patient PBMCs with DMF

We establish an ex-vivo assay to determine if DMF reduces pyroptosis and Gasdermin D activation in COVID-19 patients with severe ARDS. PBMCs will be isolated from blood and plated for 6 hours. Following a resting phase PBMCs are treated with DMF with 2 hrs. Supernatants and cell lysates are collected and subjected to the assays outlined above.

### Example 2.3: Assessment of DMF in a COVID-19 Mouse Model

We establish a mouse model of COVID-19 to determine the efficacy of DMF in suppressing Gasdermin D dependent lung inflammation in vivo. hACE2 expression is achieved through delivery in a lung trophic AAV-vector. The UMMS Gene Therapy Viral Vector Core has generated AAV-hACE2 for use in these studies. Mice are infected with AAV-hACE2 (which delivers stable expression of ACE2 to the lung). After 1 month of rest post AAV treatment these mice are infected with SARS-CoV2 at 50, 100 and 200 PFU/mouse to determine the LD50. Mice are monitored for weight loss and survival over a 21-day period. At one-week intervals serum and lung tissue are collected. Lung tissue is also assessed histologically for signs of inflammation. Mice are also examined by Barometric plethysmography and their Respiratory Physiology examined via FlexiVent in the Small animal respiratory physiology Core at UMMS. In addition, levels of IL-1 and IL-18 in the lung tissue and serum are measured by ELISA. In addition, activation status of GasderminD is determined in lung tissue lysates by western blotting. Viral plaque assays are also performed to determine viral load during the course of infection. Determining the key time points of Gasdermin D activation and pyroptosis during SARS-Cov2 infection allows dosing with DMF at the appropriate time to maximize therapeutic benefit without allowing viral replication to expand as a result of an anti-inflammatory tissue environment. Mice receive daily doses of DMF (100 mg/kg) via oral gavage starting 14-days post infection for 7 days. At day 21 lung tissue and serum are collected and assessed as outline above.

### Example 3. Fumarate-Induced Succination of Gasdermin D for the Treatment of Familial Mediterranean Fever (FMF)

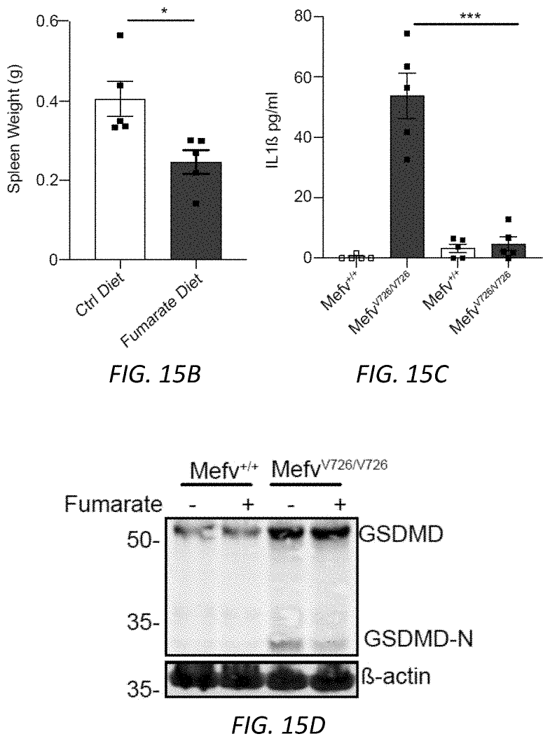

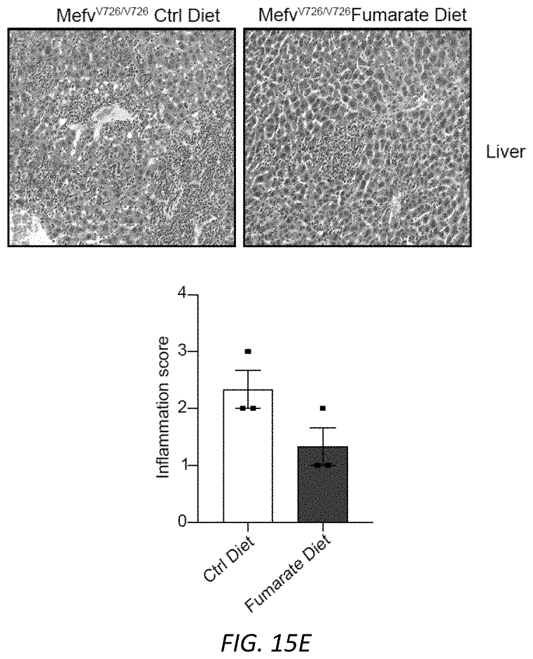

GSDMD has also been linked to familial Mediterranean-fever (FMF). FMF results from constitutive activation of the pyrin inflammasome and mice harboring the Mefv^{V726/726} allele exhibit features of the human disease. GSDMD deficient mice are rescued from disease in this model (19). Administration of fumarate alleviated weight loss, splenomegaly, IL-1β secretion, GSDMD-N formation and liver pathology in the Mefv^{V726/V726} mouse model (FIG. 15A-E). Specifically, oral administration of a diet including 0.1% DMF resulted in increased weight (FIG. 15A) and rescue of runting (FIG. 15B), reduced spleen size (FIG. 15C), reduced levels of GsdmD-p30 (and reduced pyroptosis, FIG. 51D), and reduction in fibrosis of liver and lung (FIG. 15E, F).

### REFERENCES

1. J. Shi et al., Cleavage of GSDMD by inflammatory caspases determines pyroptotic cell death. Nature 526, 660-665 (2015).
2. N. Kayagaki et al., Caspase-11 cleaves gasdermin D for non-canonical inflammasome signalling. Nature 526, 666 (2015).
3. E. L. Mills et al., Itaconate is an anti-inflammatory metabolite that activates Nrf2 via alkylation of KEAP1. Nature 556, 113-117 (2018).
4. S.-T. Liao et al., 4-Octyl itaconate inhibits aerobic glycolysis by targeting GAPDH to exert anti-inflammatory effects. Nature Communications 10, 5091 (2019).
5. V. Lampropoulou et al., Itaconate Links Inhibition of Succinate Dehydrogenase with Macrophage Metabolic Remodeling and Regulation of Inflammation. Cell Metabolism 24, 158-166 (2016).
6. M. Bambouskova et al., Electrophilic properties of itaconate and derivatives regulate the IκBζ-ATF3 inflammatory axis. Nature 556, 501-504 (2018).
7. G. M. Tannahill et al., Succinate is an inflammatory signal that induces IL-1β through HIF-1α. Nature 496, 238-242 (2013).
8. M. N. J. Jibran Sualeh Muhammad, Noha Mousaad Elemam, Thenmozhi Venkatachalam, Tom Kalathil Raju, Rifat Akram Hamoudi, Azzam A Maghazachi Gasdermin D Hypermethylation Inhibits Pyroptosis And LPS-Induced IL-1β Release From NK92 Cells. Immunotargets Ther. 8, 29-41 (2019).
9. M. Blatnik, N. Frizzell, S. R. Thorpe, J. W. Baynes, Inactivation of Glyceraldehyde-3-Phosphate Dehydrogenase by Fumarate in Diabetes. Formation of S-(2-Succinyl)Cysteine, a Novel Chemical Modification of Protein and Possible Biomarker of Mitochondrial Stress 57, 41-49 (2008).
10. M. D. Kornberg et al., Dimethyl fumarate targets GAPDH and aerobic glycolysis to modulate immunity. Science 360, 449-453 (2018).
11. A. Singh et al., Small Molecule Inhibitor of NRF2 Selectively Intervenes Therapeutic Resistance in KEAP1-Deficient NSCLC Tumors. ACS Chemical Biology 11, 3214-3225 (2016).
12. M. Kato, K. Sakai, A. Endo, Koningic acid (heptelidic acid) inhibition of glyceraldehyde-3-phosphate dehydrogenases from various sources. Biochimica et Biophysica Acta (BBA)—Protein Structure and Molecular Enzymology 1120, 113-116 (1992).
13. R. A. Kulkarni et al., A chemoproteomic portrait of the oncometabolite fumarate.

Nature Chemical Biology 15, 391-400 (2019).
14. X. Liu et al., Inflammasome-activated gasdermin D causes pyroptosis by forming membrane pores. Nature 535, 153-158 (2016).
15. J. J. Hu et al., FDA-approved disulfiram inhibits pyroptosis by blocking gasdermin D pore formation. Nature Immunology 21, 736-745 (2020).
16. C. Rogers et al., Gasdermin pores permeabilize mitochondria to augment caspase-3 activation during apoptosis and inflammasome activation. Nature Communications 10, 1689 (2019).
17. B. A. McKenzie et al., Caspase-1 inhibition prevents glial inflammasome activation and pyroptosis in models of multiple sclerosis. Proceedings of the National Academy of Sciences 115, E6065-E6074 (2018).
18. S. Li et al., Gasdermin D in peripheral myeloid cells drives neuroinflammation in experimental autoimmune encephalomyelitis. The Journal of Experimental Medicine, jem.20190377 (2019).
19. A. Kanneganti et al., GSDMD is critical for autoinflammatory pathology in a mouse model of Familial Mediterranean Fever. The Journal of Experimental Medicine 215, 1519-1529 (2018).
20. J. K. Rathkey et al., Chemical disruption of the pyroptotic pore-forming protein gasdermin D inhibits inflammatory cell death and sepsis. Science Immunology 3, eaat2738 (2018).
21. G. Sollberger et al., Gasdermin D plays a vital role in the generation of neutrophil extracellular traps. Science Immunology 3, eaar6689 (2018).
22. P. Orning, D. Weng, K. Starheim, D. Ratner, Z. Best, B. Lee, A. Brooks, S. Xia, H. Wu, M. A. Kelliher, S. B. Berger, P. J. Gough, J. Bertin, M. M. Proulx, J. D. Goguen, N. Kayagaki, K. A. Fitzgerald, E. Lien, Pathogen blockade of TAK1 triggers caspase-8-dependent cleavage of gasdermin D and cell death. Science 362, 1064-1069 (2018).
23. J. J. Chae, Y.-H. Cho, G.-S. Lee, J. Cheng, P. P. Liu, L. Feigenbaum, S. I. Katz, D. L. Kastner, Gain-of-function Pyrin mutations induce NLRP3 protein-independent interleukin-1p activation and severe autoinflammation in mice.

Immunity 34, 755-768 (2011).
24. R. Dutta, K. R. Mahajan, K. Nakamura, D. Ontaneda, J. Chen, C. Volsko, J. Dudman, E. Christie, J. Dunham, R. J. Fox, B. D. Trapp, Comprehensive Autopsy Program for Individuals with Multiple Sclerosis. J. Vis. Exp. 149, e59511 (2019).
25. S. Rühl et al., ESCRT-dependent membrane repair negatively regulates pyroptosis downstream of GSDMD activation. Science 362, 956-960 (2018).
26. K. Yuki, M. Fujiogi, S. Koutsogiannaki, COVID-19 pathophysiology: A review. Clinical Immunology, 108427 (2020).
27. C. Huang et al., Clinical features of patients infected with 2019 novel coronavirus in Wuhan, China. The Lancet 395, 497-506 (2020).
28. Y. Zhou et al., Pathogenic T cells and inflammatory monocytes incite inflammatory storm in severe COVID-19 patients. National Science Review, (2020).
29. C. Qin et al., Dysregulation of immune response in patients with COVID-19 in Wuhan, China. Clinical Infectious Diseases, (2020).
30. Y. Han et al., Lactate dehydrogenase, a Risk Factor of Severe COVID-19 Patients. medRxiv, 2020.2003.2024.20040162 (2020).
31. T. Dolinay et al., Inflammasome-regulated Cytokines Are Critical Mediators of Acute Lung Injury. American Journal of Respiratory and Critical Care Medicine 185, 1225-1234 (2012).
32. P. Broz, V. M. Dixit, Inflammasomes: mechanism of assembly, regulation and signalling. Nature Reviews Immunology 16, 407 (2016).
33. N. Kayagaki et al., Non-canonical inflammasome activation targets caspase-11.

Nature 479, 117 (2011).
34. X. Liu et al., Inflammasome-activated gasdermin D causes pyroptosis by forming membrane pores. Nature 535, 153 (2016).
35. J. Shi et al., Cleavage of GSDMD by inflammatory caspases determines pyroptotic cell death. Nature 526, 660-665 (2015).
36. N. E. B. Saidu et al., Dimethyl fumarate, a two-edged drug: Current status and future directions. Medicinal Research Reviews 39, 1923-1952 (2019).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject who has an inflammatory condition, the method comprising administering to the subject an effective amount of (i) fumarate or a fumarate analog, or (ii) an inhibitor of fumarate hydratase (FH).

2. The method of claim 1, wherein the inflammatory condition is associated with or a result of a viral infection.

3. The method of claim 2, wherein the viral infection is infection with SARS-CoV-2.

4. The method of claim 1, wherein the inflammatory condition is septic shock, autoimmune disease, allergy, asthma, cancer, celiac disease, glomerulonephritis, hepatitis, inflammatory bowel disease, preperfusion injury, transplant rejection, myalgic encephalomyelitis/chronic fatigue syndrome, neonatal-onset multisystem inflammatory disease (NOMID), or Familial Mediterranean Fever (FMF).

5. The method of claim 4, wherein the autoimmune disease is multiple sclerosis.

6. The method of claims 1-5, wherein the fumarate or fumarate analog is Dimethyl Fumarate, Diroximel Fumarate, or Tepilamide Fumarate.

7. The method of claims 1-5, wherein the inhibitor of FH is a small molecule inhibitor or an inhibitory nucleic acid targeting a nucleic acid encoding FH.

8. The method of claim 7, wherein the inhibitory nucleic acid is an antisense RNA, antisense DNA, chimeric antisense oligonucleotides, antisense oligonucleotides comprising modified linkages, interference RNA (RNAi), short interfering RNA (siRNA), or a short hairpin RNA (shRNA).

9. A composition comprising (i) fumarate or a fumarate analog, or (ii) an inhibitor of fumarate hydratase (FH), for use in a method of treating a subject who has an inflammatory condition.

10. The composition for the use of claim 9, wherein the inflammatory condition is associated with or a result of a viral infection.

11. The composition for the use of claim 10, wherein the viral infection is infection with SARS-CoV-2.

12. The composition for the use of claim 8, wherein the inflammatory condition is septic shock, autoimmune disease, allergy, asthma, cancer, celiac disease, glomerulonephritis, hepatitis, inflammatory bowel disease, preperfusion injury, transplant rejection, myalgic encephalomyelitis/chronic fatigue syndrome, neonatal-onset multisystem inflammatory disease (NOMID), or Familial Mediterranean Fever (FMF).

13. The composition for the use of claim 12, wherein the autoimmune disease is multiple sclerosis.

14. The composition for the use of claims 9-13, wherein the fumarate or fumarate analog is Dimethyl Fumarate, Diroximel Fumarate, or Tepilamide Fumarate.

15. The composition for the use of claims 9-13, wherein the inhibitor of FH is a small molecule inhibitor or an inhibitory nucleic acid targeting a nucleic acid encoding FH.

16. The composition for the use of claim 15, wherein the inhibitory nucleic acid is an antisense RNA, antisense DNA, chimeric antisense oligonucleotides, antisense oligonucleotides comprising modified linkages, interference RNA (RNAi), short interfering RNA (siRNA), or a short hairpin RNA (shRNA).
